SEQUENCE LISTING (21 sequences)

SEQ ID NO: 1 - DNA, 40 bp, Artificial Sequence
  Description of Artificial Sequence Synthetic oligonucleotide
gggaacttgacctcacagaacgtttatagtcgtttaaatg

SEQ ID NO: 2 - DNA, 37 bp, Artificial Sequence
  Description of Artificial Sequence Synthetic oligonucleotide
aggccatggccaatttttgccggcgataaaaaggtag

SEQ ID NO: 3 - DNA, 53 bp, Artificial Sequence
  Description of Artificial Sequence Synthetic oligonucleotide
ttggccccatttaaatgaatcgtttgtgttatgtttcaacgtgtttatttttc

SEQ ID NO: 4 - DNA, 61 bp, Artificial Sequence
  Description of Artificial Sequence Synthetic oligonucleotide
aattgaaaaataaacacgttgaaacataacacaaacgattcatttaaatggggccaatatt

SEQ ID NO: 5 - DNA, 57 bp, Artificial Sequence
  Description of Artificial Sequence Synthetic oligonucleotide
ggcaggtggaggccgttcaggcggaggtggctctggcggtggcggatccggggattt

SEQ ID NO: 6 - DNA, 57 bp, Artificial Sequence
  Description of Artificial Sequence Synthetic oligonucleotide
aaatccccggatccgccaccgccagagccacctccgcctgaaccgcctccacctgcc

SEQ ID NO: 7 - DNA, 36 bp, Artificial Sequence
  Description of Artificial Sequence Synthetic oligonucleotide
gatctagaggatcgcatcaccatcaccatcactaat

SEQ ID NO: 8 - DNA, 32 bp, Artificial Sequence
  Description of Artificial Sequence Synthetic oligonucleotide
attagtgatggtgatggtgatgcgatcctcta

SEQ ID NO: 9 - DNA, 27 bp, Artificial Sequence
  Description of Artificial Sequence Synthetic oligonucleotide
ccctcatgaagcgcgcaagaccgtctg

SEQ ID NO: 10 - DNA, 27 bp, Artificial Sequence
  Description of Artificial Sequence Synthetic oligonucleotide
cccaagcttagtgatggtgatggtgat

SEQ ID NO: 11 - protein, 8 aa, Artificial Sequence
  Description of Artificial Sequence Synthetic peptide
GNTLSQNV

SEQ ID NO: 12 - protein, 26 aa, Artificial Sequence
  Description of Artificial Sequence Synthetic peptide
VYSRLNEIDTKQTTVESDISAIKTSI

SEQ ID NO: 13 - protein, 361 aa, Artificial Sequence
  Description of Artificial Sequence Synthetic fiber-fibritin-6His chimera
MKRARPSEDTFNPVYPYDTETGPPTVPFLTPPFVSPNGFQESPPGVLSLRLSEPLVTSNGMALKMGNGLSLDEAGNLTSQNVYSRLNEIDTKQTTVESDISAIKTSIGYPGNNSIITSVNTNTDNIASINLELNQSGGIKQRLTVIETSIGSDDIPSSIKGQIKDNTTSIESLNGIVGENTSSGLRANVSWLNQIVGTDSSGGQPSPPGSLLNRVSTIETSVSGLNNDVQNLQVEIGNNSTGIKGQVVALNTLVNGTNPNGSTVEERGLTNSIKANETNIASVTQEVNTAKGNISSLQGDVQALQEAGYIPEAPRDGQAYVRKDGEWVLLSTFLSPAGGGGSGGGGSGGGGSRGSHHHHHH

SEQ ID NO: 14 - protein, 7 aa, Artificial Sequence
  Description of Artificial Sequence Synthetic peptide
RGDCCCC

SEQ ID NO: 15 - protein, 6 aa, Artificial Sequence
  Description of Artificial Sequence Synthetic peptide motif
FNPVYD

SEQ ID NO: 16 - protein, 9 aa, Artificial Sequence
  Description of Artificial Sequence Synthetic peptide
RGSHHHHHH

SEQ ID NO: 17 - protein, 6 aa, Artificial Sequence
  Description of Artificial Sequence Synthetic 6xHis tag
HHHHHH

SEQ ID NO: 18 - protein, 4 aa, Artificial Sequence
  Description of Artificial Sequence Synthetic peptide
SQNV

SEQ ID NO: 19 - protein, 5 aa, Artificial Sequence
  Description of Artificial Sequence Synthetic peptide
HHHHH

SEQ ID NO: 20 - protein, 4 aa, Artificial Sequence
  Description of Artificial Sequence Synthetic peptide
GLNT

SEQ ID NO: 21 - DNA, 36083 bp, Artificial Sequence
  Description of Artificial Sequence Synthetic nucleotide sequence
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcactctaggcggccgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgagctatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatccggtcgcgcgaattgatccaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaatcaaatacaaaggatatcaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatcctccgaggcctcgacctgcaggcatgcaagcttgggatctttgtgaaggaaccttacttctgtggtgtgacataattggacaaactacctacagagatttaaagctctaaggtaaatataaaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcctagagggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccgggaacttgacctcacagaacgtttatagtcgtttaaatgaaattgacactaaacagacaacagttgagtctgacattagtgctattaagacctcaataggatatccaggaaataattcgattatcacgagtgttaatacaaacactgataatattgcatctattaatttagagctaaatcaaagtggaggtattaaacagcgtttaaccgttattgaaacttccattggttcagatgatattccttcgagtattaaaggtcaaatcaaagataatacaacttcaatcgaatctctaaatggaatcgtcggtgaaaacacttcatctggcttaagagcgaatgtttcatggttaaaccaaattgttggaactgattctagcggtggacaaccttctcctcctgggtctcttttaaaccgagtttctacaattgaaacttctgtttcaggcttgaataacgatgttcaaaacctacaagtagagattggtaataacagcacaggaattaaagggcaagttgtagcgttaaatactttagtaaatggaactaatccaaacggttcaactgttgaagagcgcggattaaccaattcaataaaagctaacgaaactaacattgcatcagttacacaagaagtgaatacagctaaaggcaatatatcttctttacaaggtgatgttcaagctctccaagaagccggttatattcctgaagctccaagagatgggcaagcttacgttcgtaaagatggcgaatgggtattgctttctacctttttatcgccggcaggtggaggcggttcaggcggaggtggctctggcggtggcggatccgctatcgttatgacccagccgagttctctgtctgtatcactgggtgaacgtgttaccatcagctgccgcgcgtcccagagcctggaaaacagcaatggtaacaccttcctgaattggtatcagcaaaagcctggccaaccgccgaaactgctgatctaccgtgtttctaaccgtttctctggtgttccggaccgtttctcgggtagcggcagcggtaccgacttcactctgaccatctcttctgtacaggctgaagatctggctgtttactactgtctgcaagttacccacgtaccgtacacctttggcgctggcaccaaactggagctcaagggtggcggtggctcgggcggtggtgggtcgggtggcggcggatctgaagttaaactgcagcagagcggggctgagcttgtgaagcccgggggttcagtgaagctgtcctgcaaggcttctggctacaccttcaccaccaactacaattggaactgggtgaagcagaggcctggacaaggtctcgagtggattggatacattcgttatgacggtacctctgaatacaccccgtccctgaagaacaaggccacactgactgtagacaaatcgtccagcacagcctacatgcagctcagcagcctgacatctgaggactctgcggtctattattgtgctcgtctggactactggggccaagggaccacgctcaccgtctcctcacatcatcaccatcaccactaataaatgaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

(19) United States
(12) Patent Application Publication
(10) Pub. No.: US 2005/0095231 A1
(43) Pub. Date: May 5, 2005

(54) Modified adenovirus containing a fiber replacement protein

(76) Inventors: David T. Curiel, Birmingham, AL (US); Nikolay Korokhov, Birmingham, AL (US)

Correspondence Address: FROMMER LAWRENCE & HAUG, 745 FIFTH AVENUE- 10TH FL., NEW YORK, NY, 10151, US

(21) Appl. No.: 10/944,496

(22) Filed: Sep. 17, 2004

Related U.S. Application Data
(63) Continuation-in-part of application No. 09/612,852, filed on Jul. 10, 2000, now Pat. No. 6,815,200.
Application No. 09/612,852 is a continuation-in-part of application No. 09/250,580, filed on Feb. 16, 1999, now Pat. No. 6,210,946.
(60) Provisional application No. 60/074,844, filed on Feb. 17, 1998.

(51) Int. Cl. A61K 48/00; C12N 7/00; C12N 5/08; C12N 15/861

(52) U.S. Cl. 424/93.21; 435/456; 435/366; 435/235.1

(57) ABSTRACT

The utility of adenovirus vectors (Ad) for gene therapy is restricted by their inability to selectively transduce disease-affected tissues. This limitation may be overcome by the derivation of vectors capable of interacting with receptors specifically expressed in the target tissue. Previous attempts to alter Ad tropism by genetic modification of the Ad fiber have had limited success due to structural conflicts between the fiber and the targeting ligand. The present invention presents a strategy to derive an Ad vector with enhanced targeting potential by a radical replacement of the fiber protein in the Ad capsid with a chimeric molecule containing a heterologous trimerization motif and a stabilized scFv ligand.

### INCORPORATION BY REFERENCE

This continuation-in-part application claims benefit of U.S. application Ser. No. 09/612,852 filed Jul. 10, 2000, which is a continuation-in-part application of U.S. application Ser. No. 09/250,580 filed Feb. 16, 1999, now U.S. Pat. No. 6,210,946 issued Apr. 3, 2001, which claims benefit of U.S. provisional application Ser. No. 60/074,844 filed Feb. 17, 1998.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FEDERAL FUNDING LEGEND

This invention was supported in part using federal funds from the National Institutes of Health. Accordingly, the Federal Government has certain rights in this invention.

### FIELD OF THE INVENTION

The present invention relates generally to the fields of vector biology and gene therapy. More specifically, the present invention relates to the production of recombinant adenoviral vectors with replacement of fibers for cell-specific targeting with concomitant elimination of endogenous tropism.

### BACKGROUND OF THE INVENTION

Approaches to target adenoviral vectors to specific cell types should be based on an understanding of the mechanism of cell entry exploited by the majority of human adenoviruses and on the identification of the components of the adenoviral virion which are involved in the early steps of the virus-cell interaction. Adenoviruses are non-enveloped viruses containing a double stranded DNA genome packaged into an icosahedral capsid. Whereas the most abundant capsid protein, the hexon, performs structural functions and is not involved in the active cell entry process, the other two major protein components of the capsid, the fiber and the penton base, have been shown to play key roles in the early steps of virus-cell interaction. The fiber and penton base together form penton capsomers consisting of five penton base subunits embedded in the virus capsid tightly associated with a homotrimer of fiber proteins protruding from the virion.

Each of the five subunits of the penton base contains a flexible loop structure, which corresponds to a hypervariable domain of the otherwise highly conserved protein. Amino acid sequence analysis of penton base proteins of different adenoviral serotypes showed that each loop consists of two stretches of alpha helices flanking an arginine-glycine-aspartic acid (RGD) tripeptide positioned in the middle of the loop. Cryo-electron micrography (cryo-EM) studies of Ad2 virions revealed that these loops form 22A protrusions on the surface of penton base, thereby facilitating interaction of the RGD motif, localized at the apex of the protrusion with cellular integrins.

The fiber has a well-defined structural organization with each of its three domains, the tail, the shaft, and the knob, performing a number of functions vital for the virus. The short amino terminal tail domain (46 amino acid residues in Ad2 and Ad5 fibers) of the fiber protein is highly conserved among most adenoviral serotypes. In addition to being involved in the association with the penton base protein through an FNPVYD (SEQ ID NO:15) motif at residues 11-16, which results in anchoring the fiber to the adenoviral capsid, the tail domain also contains near its amino terminus the nuclear localization signal KRλR (where λ indicates a small amino acid residue), which directs the intracellular trafficking of newly synthesized fibers to the cell nucleus, where the assembly of the adenoviral particle takes place.

The central domain of the fiber is the shaft, which extends the carboxy terminal knob domain away from the virion, thereby providing optimal conditions for receptor binding. The shaft is organized as a sequence of pseudorepeats, each 15 amino acids in length, with a characteristic consensus sequence containing hydrophobic residues at highly conserved positions. This sequence, X-X-φ-X-φ-X-φ-G-X-G-φ-X-φ-X-X or X-X-φ-X-φ-X-φ-X-X-P-φ-X-φ-X-X, contains hydrophobic amino acids at “φ”-positions, with either the eighth and tenth positions being occupied with two glycines or with a proline in the tenth position. The models for the secondary structure corresponding to these repeats describe the shaft as a triple β-spiral in which the β-strands are oriented more along the fiber axis and the hydrophobic residues at the 7^{th} and 13^{th} position are located at greater radius. The trimer is stabilized with extensive intra- and inter-chain hydrogen bonding. Due to its rod-like shape, the shaft domain basically determines the length of the entire molecule, which depends on the number of pseudorepeats contained within the shaft. The fibers of various human adenoviral serotypes contain different number of repeats, resulting in a significant variation in the fiber length: from 160A (Ad3) to 373A (Ad2 and AM).

The carboxy terminal knob domain (180-225 amino acid residues) carries out two distinct functions, i.e., initiation of fiber trimerization and binding of the virus to its primary cellular receptor. X-ray crystallography studies on E. coli-expressed Ad5 fiber knob protein have shown that the trimeric knob is arranged around a three-fold crystallographic symmetry axis and resembles a three bladed propeller when viewed along this axis. Each monomer of the knob is a β-sandwich structure, formed by two antiparallel β-sheets R and V. The surface of the V-sheet, which consists of the strands A, B, C, and J, points towards the virion, while the R-sheet, formed by strands D, I, H, and G, points outside the virion and towards the surface of the target cell. These findings have been then corroborated with X-ray crystallography data obtained with recombinant Ad2 fiber knob protein.

A number of studies employing recombinant knobs have shown that these proteins are capable of self-trimerization, which does not require any cellular chaperons. The exact trimerization motif within the fiber knob is largely unknown, which makes mutagenesis or modification of this protein quite difficult: indeed, any new mutation or modification of the fiber may affect amino acid(s) involved in the fiber trimerization and may therefore destabilize the entire molecule, thereby rendering it non-functional. The mutant knobs revealed that deletions in the knob sequence, even as short as two amino acid residues, may result in monomeric fibers, which cannot associate with penton base and, therefore, cannot be incorporated into mature adenoviral particles.

The second function performed by the knob is binding to a cellular receptor and, therefore, mediating the very first step of the virus-cell interaction. This receptor-binding ability of the knob has been demonstrated by utilization of recombinant knob proteins as specific inhibitors of adenoviral binding to cells. Based on the β-sandwich structure of the knob, it was originally hypothesized by Xia et al. that the strands constituting the R-sheet form a receptor binding structure. Recently, however, analysis of fiber knob mutants has revealed that segments outside the R-sheet constitute the receptor-binding site. The Ad5 binding site is located at the side of the knob monomer and specifically involves sequences within the AB and DE loops and B, E, and F β-strands. The binding site of Ad37 that binds to a different receptor involves a critical residue in the CD loop at the apex of the trimer.

The two penton proteins, the penton base and fiber, work in a well-orchestrated manner to provide the early steps of the cell infection mechanism developed by adenoviruses. Importantly, each of these early events is mediated by either fiber or penton base; therefore, both proteins play distinct and well defined roles in this process.

The fiber knob provides the initial high-affinity binding of the virus to its cognate cell surface receptor, coxsackievirus and adenovirus receptor (CAR), which does not possess any internalization functions and merely works as a docking site for Ad attachment.

Human adenoviruses (Ad) of serotype 2 and 5 have been extensively used for a variety of gene therapy applications. This is largely due to the ability of these vectors to efficiently deliver therapeutic genes to a wide range of different cell types. However, the promiscuous tropism of adenovirus resulting from the widespread distribution of coxsackie virus and adenovirus receptor (CAR) (Bergelson et al., Science 275, 1320-3 (1997) and Tomko et al., Proc. Natl. Acad. Sci. 94, 3352-6 (1997)), limits the utility of adenoviral vectors in those clinical contexts where selective delivery of therapeutic transgene to a diseased tissue is required. Uncontrolled transduction of normal tissues with adenoviral vectors expressing potentially toxic gene products may lead to a series of side effects, thereby undermining the efficacy of the therapy. Furthermore, cell targets expressing CAR below certain threshold levels are not susceptible to adenoviralbased therapies due to their inability to support adenoviral infection. Therefore, the dependence of the efficiency of the adenoviralmediated cell transduction on the levels of CAR expression by the target cell presents a serious challenge for the further development of adenoviral-based gene therapeutics.

In order to overcome this limitation, the concept of genetic targeting of adenoviral vectors to specific cell surface receptors has been proposed. Strategies to retarget adenoviral vectors are based on the currently accepted model of adenoviral infection (Krasnykh et al., Molecular Therapy 1, 391-405 (2000)), which postulates that the initial binding of the adenoviral virion to the cell is mediated by the attachment of the globular knob domain of the adenoviral fiber protein to CAR. This is then followed by an internalization step triggered by the interaction of the RGD-containing loop of a second adenoviral capsid protein, the penton base, with cellular integrins. Although recent studies have shown that representatives of different adenoviral serotypes may utilize cell receptors other than CAR, the two-step mechanism of cell entry established for Ad2 and Ad5 appears to be common to the majority of human adenovirus. As the fiber protein is the key mediator of the cell attachment pathway employed by Ad, genetic incorporation of targeting ligands within this viral protein was originally proposed as the strategy to derive targeted, cell type specific adenoviral vectors.

Although the primary amino acid sequences of fiber proteins of various human and animal adenoviruses are highly diverse, the overall structural and functional organization of these proteins demonstrate remarkable degree of similarity. Indeed, all key features of the domains of the fiber proteins described above—the presence of the nuclear localization signal and the penton base binding site within the fiber tail; the presence of pseudorepeats in the shaft; the propeller-like structure of the knob; and trimeric configuration of the entire fiber molecule—are highly conserved between various adenoviral serotypes. This overall structural and functional similarity has been exploited by a number of investigators, who succeeded in replacing the entire fiber proteins of one adenoviral serotype with those derived from another serotype, or “shuffled” individual domains of the fiber molecule utilizing a variety of structural domains pre-existing in nature.

However, it is of paramount importance to note that fiber shuffling does not overcome the limitations associated with the conserved structure of native fibers: as all the adenoviral fibers characterized so far contain the knob domains of similar structure, which carry out the functions of trimerization and receptor binding, it is logical to assume that replacing those knobs with their structurally similar counterparts derived from other adenoviral serotypes would lead to chimeric molecules inheriting all the drawbacks and structural limitations known for the wild type fibers in the context of incorporation of the cell-targeting ligands within these carrier proteins. The same holds true with respect to shuffling of the full size fibers.

In addition, as all wild type adenoviral fibers have affinity to their cognate receptors, it is rather problematic to create recombinant adenoviral vectors targeted to specific cell surface receptors via the fiber shuffling. This maneuver may change the tropism of the vector, but will never result in an adenoviral vector specifically targeted to the cell of interest. Although ablation of native tropism of adenoviral vector via identification and subsequent elimination of specific amino acids of the fiber protein which mediate binding of the virion to its native receptor is generally viewed as the way of derivation of truly targeted adenoviral vectors, it may have limited utility as the mutated sequences may undergo reversion to the wild type during multiple cycles of virus propagation. Due to its restored ability to bind to its native receptor a virion which genome underwent such a reversion immediately achieves selective advantage over the virions which tropism is restricted to one specific receptor. This selective advantage will eventually result in significant contamination of the vector preparation with virions retaining tropism to receptors different from the target one. Therefore the efficiency of the entire targeting maneuver will be jeopardized.

Furthermore, many human adenoviruses recognize CAR as the primary binding receptor which is expressed by many different cell types. Taken together with the widespread distribution of adenoviral infections in humans, this has led to the belief that chimeric adenoviral virions incorporating fiber proteins originating from different adenoviral serotypes most likely exist in nature when the same cell in a human body gets infected with two adenoviruses belonging to two different Ad serotypes. Therefore, shuffling the fibers is an experimental realization of the viral chimerizm which takes place naturally.

Attempts to generate adenoviral vectors possessing expanded tropism involved incorporation of short peptide ligands into either the carboxy terminal or so-called HI loop of the knob of the Ad fiber protein. Although these studies demonstrated the feasibility of genetic targeting of Ad and showed the potential utility of such vectors in the context of several disease models (Vanderkwaak et al., Gynecol Oncol 74, 227-34 (1999) and Kasono et al., Clinical cancer research 5, 2571-2579 (1999)), further progress in this direction has been hampered by the structural conflicts often observed as a result of modification of the fiber structure. Due to the rather complex structure of the fiber knob domain, even minor modifications to this portion of the molecule may destabilize the fiber, thereby rendering it incapable of trimerization and, hence, non-functional. The upper size limit for a targeting ligand to be incorporated into Ad5 fiber is about 30 amino acid residues (Wickham et al., Journal of Virology 71, 8221-8229 (1997) and Hong and Engler, J Virol 70, 7071-8 (1996)), which dramatically narrows the repertoire of targeting moieties, thereby limiting the choice of potential ligands and, therefore, cell targets. The task of adenoviral targeting is further complicated by the need to ablate the native receptor-binding sites within the fiber of an adenoviral vector to make it truly targeted. As a result of these limitations, only a handful of heterologous peptide ligands (oligo lysine, FLAG, RGD-4C (SEQ ID NO: 14), RGS(His)₆ (SEQ ID NO: 16), and HA epitope) have been successfully used in the context of Ad5 fiber modification during last several years.

The prior art remains deficient in the lack of effective means to produce recombinant adenoviral vectors with combination of novel targeting and ablation of native tropism. The present invention fulfills this longstanding need and desire in the art.

Citation or identification of any document in this application is not an admission that such document is available as prior art to the present invention.

### SUMMARY OF THE INVENTION

The present invention describes the next generation of recombinant, cell-specific adenoviral vectors. More particularly, the instant specification discloses that there are two aspects to consider in the modification of adenoviral tropism: (1) ablation of endogenous tropism; and (2) introduction of novel tropism. To expand the utility of recombinant adenoviruses for gene therapy applications, methods to alter native vector tropism to achieve cell-specific transduction are necessary. To achieve such targeting, the present invention discloses the development of a targeted adenovirus created by radical replacement of the adenovirus fiber protein. The fiber protein was replaced with a heterologous trimerization motif to maintain trimerization of the knobless fiber and a ligand capable of targeting the virion to a novel receptor was introduced simultaneously. The present invention thus represents a demonstration of the retargeting of a recombinant adenoviral vector via a non-adenoviral cellular receptor.

The invention is based, in part on Applicant's development of an adenoviral vector targetable via a stabilized scFv ligand incorporated into the capsid via the fiber replacement approach. The adenovirus (Ad) is modified by replacing a native capsid protein fiber with a fiber replacement protein, wherein the fiber replacement protein comprises: an amino-terminal portion comprising the native capsid protein fiber amino terminus; a trimeric substitute for a fiber shaft knob of the native capsid protein fiber; and a carboxy-terminal portion comprising a stabilized single chain antibody (scFv) ligand. In one embodiment, the trimeric substitute retains trimerism when a sequence encoding the stabilized scFv ligand is incorporated into the carboxy-terminus. In another embodiment, the fiber replacement protein is soluble.

The invention also provides for several trimeric substitutes, such as, but not limited to, a T4 bacteriophage fibritin protein, a trimeric substitute comprising an isoleucine trimerization motif and a trimeric substitute comprising a neck region peptide from human lung surfactant D.

In another embodiment, the adenovirus comprises a transgene, e.g., a herpes simplex virus thymidine kinase gene.

In a preferred embodiment of the invention, the stabilized scFv ligand comprises mutations in the scFv CDR regions. In another embodiment, the stabilized scFv ligand is an anti-CD40 scFv.

The invention also encompasses viral vectors, preferably an adenoviral vector comprising the adenovirus of described herein. In one embodiment, adenovirus is operatively linked to a non-viral promoter. The invention also provides for transformed host cells comprising such vectors. In one embodiment, the vector is introduced into the cell by transfection, electroporation or transformation.

The invention also provides for a method for preparing a transformed cell expressing the adenovirus of the present invention comprising transfecting, electroporating or transforming a cell with the adenovirus to produce a transformed host cell and maintaining the transformed host cell under biological conditions sufficient for expression of the adenovirus in the host cell.

In another embodiment, the invention encompasses a method for inhibiting tumor cell growth in a subject in need thereof comprising administering to the subject in need thereof a therapeutically effective amount of the adenovirus described herein wherein the scFv ligand targets the tumor cell such that the adenovirus infects the tumor cells and thereby inhibits tumor cell growth in the subject. In one embodiment, the adenovirus further comprises a transgene. In an embodiment wherein the transgene is herpes simplex virus thymidine kinase, the method for inhibiting tumor cell growth can optionally comprise administering ganciclovir.

Other and further aspects, features, and advantages of the present invention will be apparent from the following description of the presently preferred embodiments of the invention given for the purpose of disclosure.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the generation of Ad5 fiber-T4 fibritin chimera containing targeting ligand. FIG. 1A shows the schema showing key components of the fiber-fibritin-ligand chimera and their sources. The tail of the fiber anchors the fiber-fibritin-6His chimera (SEQ ID NO: 13) in the Ad virion; a fragment of the fibritin protein provides trimerization of the molecule; while the 6His (SEQ ID NO: 17) ligand mediates binding to an artificial receptor.

FIG. 1B shows SDS-PAGE analysis of E. coli-expressed, IMAC-purified FF/6H chimeric protein. M—molecular mass protein ladder (200, 116, 97, 66 and 45 kilodaltons markers are seen), lanes 1 and 2—FF/6H protein, lanes 3 and 4—wild type Ad5 fiber. Samples in lanes 1 and 3 are denatured by boiling, which resulted in degradation of trimeric proteins to monomers, while lanes 2 and 4 contain proteins in their native trimeric configuration.

FIG. 2 shows the structure of the fiber-fibritin-6H protein chimera (SEQ ID NO: 13). The FF/6H gene assembled by overlap extension PCR encodes a 373 amino acid long protein chimera which consists of the amino terminal segment of Ad5 fiber protein genetically fused with the carboxy terminal portion of the T4 fibritin protein, followed with the linker and the 6His-containing (SEQ ID NO: 17) ligand. The beginning of the third pseudorepeat of the fiber shaft domain (GNTLSQNV) (SEQ ID NO. 11) is joined to the fibritin sequence starting with the fragment of the insertion loop (SQN) preceding the sixth coiled coil segment of the α-helical central domain of the fibritin (VYSRLNEIDTKQTTVESDISAIKTSI) (SEQ ID NO. 12). The sequence SQNV (SEQ ID NO: 18) present in the native structures of both fusion partners was chosen as the hinge between the two molecules in order to minimize potential structural conflicts between the β-spiral configuration of the fiber shaft and the triple α-helix of the central domain of the fibritin. The segments of the fibritin sequence localized between every two adjacent coiled coils are the insertion loops which provide some degree of flexibility needed for optimal ligand presentation. A peptide linker is incorporated between the carboxy terminal trimerization domain (foldon) of the fibritin and the six histidine containing ligand to extend the ligand away from the carrier protein in order to facilitate binding to the target receptor.

FIG. 3 shows the Ad-mediated gene transfer to 293/6H cells. 293/6H cells were derived by transfection of 293 cells with a recombinant plasmid expressing an artificial receptor (AR), which consists of an anti-5His (SEQ ID NO: 19) scFv genetically fused with the transmembrane domain of the PDGF receptor. Due to the presence of both CAR and AR on the surface of these cells, 293/6H are susceptible to infection by both the Ad with the wild type fibers and the Ad incorporating the FF/6H chimera. Importantly, each virus is capable of binding to only one type of receptor, CAR or AR. The progenitor cell line, 293, is refractory to Ad5LucFF/6H infection.

FIG. 4 shows the generation of Ad5LucFF/6H. The genome of the wild type Ad5 was modified by homologous DNA recombination in E. coli to contain a firefly luciferase expressing cassette in place of the E1 region, as well as the gene encoding the FF/6H chimera, which replaced the wild type fiber gene. The virus, Ad5LucFF/6H, was first rescued in 211B cells expressing the wild type Ad5 fiber. The seed stock of the virus obtained at this point contained a mixed population of Ad virions with mosaic capsids incorporating both wild type Ad5 fibers and FF/6H proteins. I n order to obtain a homogeneous population of Ad virions containing FF/6H chimeras, this stock was then used to infect 293 cells expressing the artificial receptor, 293/6H. The virus isolated from 293/6H cells was purified by double banding on a CsCl gradient.

FIG. 5 shows the analysis of Ad5LucFF/6H capsid composition. FIG. 5A shows SDS-PAGE of CsCl-purified Ad5LucFF/6H virions. Samples containing 4×10¹⁰ particles of either the wild type Ad5 (lane 1) or Ad5LucFF/6H (lane 2) were boiled in Laemmli sample buffer and fractionated on a 10% SDS-PAGE gel. Of note, the resolution of this minigel is not sufficient for separation of the fiber and protein IIIa.

FIG. 5B shows Western blot analysis of FF/6H chimeras incorporated into Ad5LucFF/6H virions. Proteins of denatured Ad5LucFF/6H virions, lane 2, were separated on a 10% SDS-PAGE gel and then probed with anti-Ad fiber tail mAb 4D2, anti 5His (SEQ ID NO: 19) mAb Penta-His and anti-fibritin mouse polyclonal antibodies. Wild type Ad5, lane 1, and Ad5LucFc6H, a virus containing fibers with carboxy terminal 6His tags (SEQ ID NO: 17), lane 3, were used as controls.

FIG. 6 shows the binding of Ad5LucFF/6H virions to Ni-NTA-agarose. Wild type Ad5 or Ad5LucFF/6H were incubated with an aliquot of Ni-NTA-resin for one hour. The matrix was pelleted by centrifugation and the supernatant was removed and then incubated with a second aliquot of Ni-NTA-agarose. Aliquots of material subsequently eluted from the resin, as well as an aliquot of the material present in the supernatant after two sequential incubations with the resin, were separated on a 10% SDS-PAGE gel and then stained (FIG. 6A) or probed with either anti-fiber tail mAb 4D2 (FIG. 6B) or with anti-5His (SEQ ID NO: 19) mAb Penta-His (FIG. 6C). Lane 1, aliquot of the virus prior to incubation with Ni-NTA-agarose; lane 2, material bound to the first aliquot of the resin; lane 3, material bound to the second aliquot of the resin; lane 4, material remaining in the supernatant after two sequential bindings to the resin. Incomplete binding of Ad5LucFF/6H virions to Ni-NTA agarose is most likely due to the small size of pores in the Sepharose CL-6B used as the matrix for manufacturing Ni-NTA-agarose. According to the manufacturer's specifications, the size of those pores does not allow protein molecules with molecular mass larger that 4 MDa to enter the pores. Thus, the Ni-NTA groups which are localized on the surface of the Sepharose particles are accessible to the 6His-tagged (SEQ ID NO: 17) virions (relatively small percentage), whereas those hidden inside the pores (the majority) are not. FIG. 7 shows the analysis of Ad5LucIF/6H genome structure. FIG. 7A shows DNA isolated from purified Ad5LucFF/6H virions subjected to restriction enzyme analysis using a number of restriction endonucleases which do not cleave the wild type fiber gene sequence but cleave the FF/6H gene. Odd-numbered lanes—control Ad5Luc1 DNA; even-numbered lanes—Ad5LucFF/6H DNA. FIG. 7B shows “diagnostic PCR” utilizing a pair of primers flanking the fiber gene in Ad5 genome employed to show the absence of the wild type fiber gene sequence in the Ad5LucFF/6H genome: lane 1, PCR product amplified from wild type Ad5 DNA; lane 2, PCR product amplified from Ad5LucFF/6H DNA; M—1 Kb ladder.

FIG. 8 shows the evaluation of the efficiency and receptor-specificity of Ad5LucFF/6H-mediated gene transfer. FIG. 8A shows gene transfer to 293 and 293/6H cells. Cells seeded in 24-well plates were infected with various doses of Ad5LucFF/6H. The minimal viral dose corresponding to a multiplicity of infection of 40 viral particles per cell, (IX), was equal to the dose of the control virus, Ad5Luc1, whereas Aci5LucFF/6H doses 10×, 100×, and 1000× contained 10-, 100-, and 1000-times the amount of the control virus, correspondingly. Twenty hours postinfection, the cells were collected, lysed, and the luciferase activity of the lysates was measured in relative light units.

FIG. 8B shows the specificity of Ad5LucFF/6H binding to the artificial receptor. 293/6H cells grown in monolayer culture were pre-incubated with various concentrations of either the truncated form of fibritin or fibritin carrying a carboxy terminal 6His tag (SEQ ID NO: 17), fibritin-6H, prior to infection with Ad5LucFF/6H. Luciferase activities detected in the lysates of infected cells twenty hours postinfection were given as percentages of the activity in the absence of blocking protein. Each data point was set in triplicates and calculated as the mean of three determinations.

FIG. 9 shows the schema of key components of the fiber-fibritin-RGD/6His chimera (SEQ ID NO: 16).

FIG. 10 shows SDS-PAGE analysis of CsCl-purified Ad5LucFF.RGD/6H virions. Samples of either the wild type Ad5 (lane 2) or Ad5LucFF.RGD/6H (lane 1) were boiled in Laemmli sample buffer and fractionated on a 10% SDS-PAGE gel.

FIG. 11 shows Western blot analysis of FF.RGD/6H chimeras incorporated into Ad5LucFF/6H virions. Proteins of denatured Ad5LucFF.RGD/6H virions, lane 2, were separated on a 10% SDS-PAGE gel and then probed with anti-Ad fiber tail mAb 4D2, anti-5His (SEQ ID NO: 19) mAb Penta-His and anti-fibritin mouse polyclonal antibodies. Wild type Ad5, lane 3, and Ad5LucFc6H, a virus containing fibers with carboxy terminal 6His tags (SEQ ID NO: 17), lane 1, were used as controls.

FIG. 12 shows the binding of Ad5LucFF.RGD/6H virions to Ni-NTA-agarose. Wild type Ad5 or Ad5LucFF.RGD/6H were incubated with an aliquot of Ni-NTA-resin. Aliquots of material subsequently eluted from the resin, as well as an aliquot of the virus prior to incubation with Ni-NTA-agarose, were separated on a 10% SDS-PAGE gel and then stained.

FIG. 13 shows restriction enzyme analysis of Ad5LucFF.RGD/6H. DNA isolated from purified Ad5LucFF.RGD/6H virions was subjected to restriction enzyme analysis using a number of restriction endonucleases. Odd-numbered lanes—control Ad5Luc 1 DNA; even-numbered lanes—Ad5LucFF.RGD/6H DNA.

FIG. 14 shows gene transfer by Ad5LucFF.RGD/6H. Cells seeded in 24-well plates were infected with various doses of Ad5LucFF.RGD/6H. The minimal viral dose corresponding to a multiplicity of infection of 40 viral particles per cell, (1×), was equal to the dose of the control virus, Ad5Luc1, whereas Ad5LucFF.RGD/6H doses 10×, 100×, and 1000× contained 10-, 100-, and 1000-times the amount of the control virus, correspondingly. Twenty hours postinfection, the cells were collected, lysed, and the luciferase activity of the lysates was measured in relative light units.

FIG. 15 shows the binding of CsCl purified virions to recombinant CD40. CsCl purified virions were tested for ability to bind to recombinant CD40. Ad.FF-G28.5 (circle) and Ad.FF-CD40L (triangle) were compared with two control viruses Ad5.Fb (diamond) and Ad5.FF-6His (square) at the virus particles/well as indicated on the graphs. Absorbance was measured at 490 nm.

FIG. 16 shows a diagram of Ad5.Luc1.FF-28.51 (SEQ ID NO: 21)

### DETAILED DESCRIPTION

In marked contrast to the strategy of replacing one Ad fiber (or one of its domains) with the fiber (or its domain) derived from a different Ad serotype, the present invention presents a n alternative approach of Ad targeting based on replacement of the native fiber in an Ad capsid with a chimeric protein, rationally designed to result in permanent ablation of native Ad receptor tropism and simultaneously offers unprecedented flexibility in the generation of novel vector tropism. This work was driven by the hypothesis that these goals may be achieved by “splitting” the functions normally performed by the knob domain of the Ad5 fiber between two different protein moieties which would replace the knob. Specifically, the knob of the fiber was replaced with a heterologous trimerization motif to maintain trimerization of the knobless fiber and a ligand capable of targeting the virion to a novel receptor was introduced simultaneously. Therefore, in marked contrast to the previous, mostly unsuccessful, attempts to fit a desired ligand into the highly complex framework of the fiber knob domain, the present invention employes a radical replacement of the fiber with a protein chimera, which allows for utilization of a virtually unlimited range of targeting protein ligands in the context of Ad vector system.

The present invention is directed to vector system that provides both a highly efficient and specific targeting of adenovirus vector for the purpose of in vivo gene delivery to predefined cell types after administration. In the recombinant adenovirus of the present invention, the adenovirus is modified by replacing the adenovirus fiber protein with a fiber replacement protein. In a preferred embodiment, the fiber replacement protein comprises: an amino-terminal portion comprising the native capsid protein fiber amino terminus; a trimeric substitute for a fiber shaft knob of the native capsid protein fiber; and a carboxy-terminal portion comprising a stabilized single chain antibody (scFv) ligand. A person having ordinary skill in this art would recognize that one may exploit a wide variety of scFvs which specifically recognize cell surface proteins unique to a particular cell type to be targeted.

The following description will allow a person having ordinary skill in this art to determine whether a putative fiber replacement protein would function as is desired in the compositions and methods of the present invention. Generally, the fiber replacement protein associates with the penton base of the adenovirus. To prevent problems of incompatibility, the aminoterminus of the chimeric protein can be genetically fused with the tail domain of the adenovirus fiber. Structurally, the fiber replacement protein is preferably a rod-like, trimeric protein. It is desirable for the diameter of the rod-like, trimeric protein to b e comparable to the native fiber protein of wild type adenovirus. It is important that the fiber replacement protein retain trimerism when a sequence encoding a targeting ligand is incorporated into the carboxy-terminus. In a preferred aspect, a representative example of a fiber replacement protein is T4 bacteriophage fibritin protein. More generally, the fiber replacement protein can be any native or chimeric protein which is capable of associating with the Ad5 penton base protein and bind to specific cell surface receptor. Other representative examples of fiber replacement proteins include, but are not limited to, gene product 9 (gp9) of bacteriophage T4, heat shock transcription factor from the yeast Kluyveromyces lactis, isoleucine trimerization motif, lymphotoxin-alpha, neck region peptide from human lung surfactant D and reovirus attachment protein α1. Preferably, the fiber replacement protein has a coiled coil secondary structure. The secondary structure provides stability because of multiple interchain interactions.

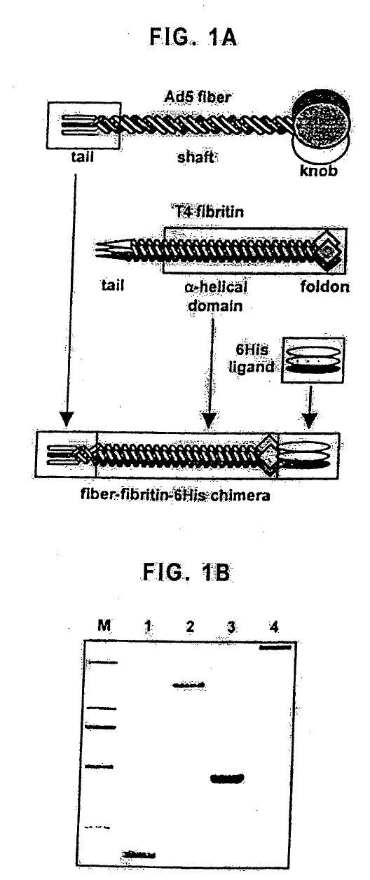

In one embodiment, the fiber-replacing molecule engineered in this study incorporated the tail and two amino terminal repeats of the shaft domain of the Ad5 fiber protein genetically fused with a truncated form of the bacteriophage T4 fibritin protein, which was employed as the heterologous trimerizing motif in order to compensate for the knob deletion (FIG. 1A). The choice of the T4 fibritin as the trimerization moiety was dictated by a number of its structural features. The fibritin protein is a product of the wac gene which forms the “collar” and the “whiskers” of the T4 capsid, where it mediates assembly of the long tail fibers and their subsequent attachment to the tail baseplate. Trimerization of this rod-like, 486 amino acid long protein is initiated and maintained b y the short (30 as long) carboxy terminal domain or “foldon”, which is stabilized by a number of hydrophobic interactions and hydrogen bonds (Tao et al., Structure 5, 789-98 (1997)). The central α-helical domain of fibritin which consists of 13 segments of parallel triple coiled-coils separated by flexible loop structures, passively follows the trimerization initiated at the carboxy terminal of the molecule. The trimeric structure of fibritin is extremely stable and is not compromised by either extensive amino terminal deletions (up to 92% of the molecule) (Letarov et al., Biochemistry (Mosc) 64, 817-23 (1999)) or carboxy terminal insertions up to, at least, 163 as long (Letarov et al., Biochemistry (Mosc) 64, 817-23 (1999) and V. V. Mesyanzhinov, personal communication). For the purposes of this study, no receptor-binding function has been shown for fibritin.

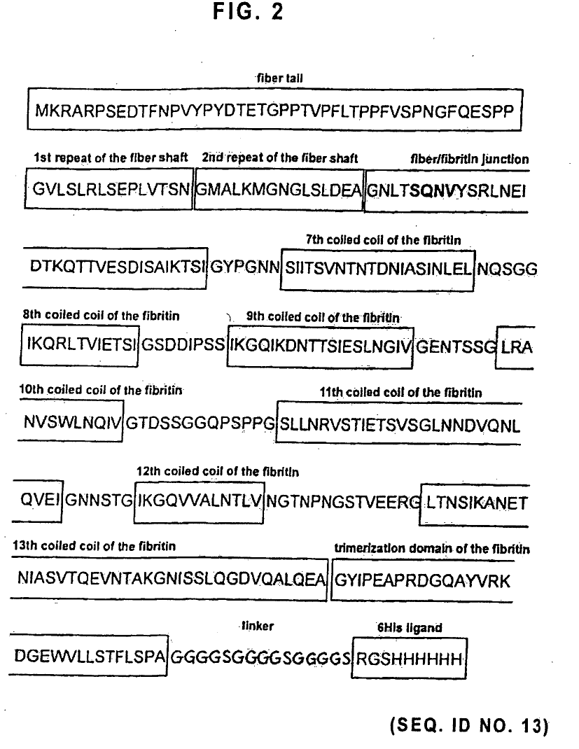

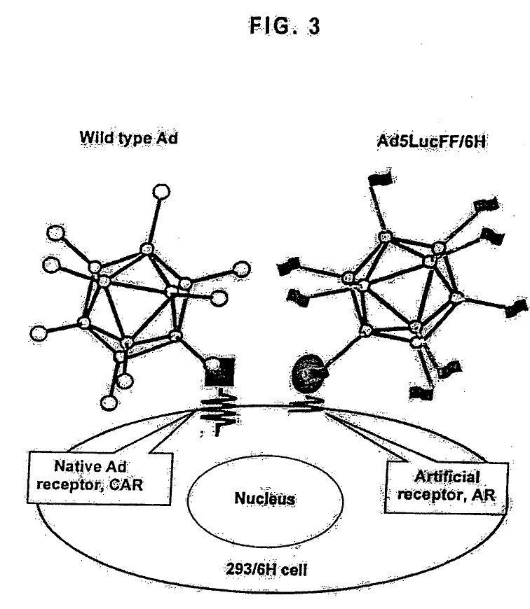

In order to provide a receptor-binding ligand, a carboxy terminal six-histidine sequence was connected to the fibritin protein of this fiber-fibritin chimera via a short peptide linker (FIG. 2). The purpose of this maneuver was to demonstrate the feasibility of targeting of fibritin-containing Ad vectors to alternative cell-surface receptors by directing the modified vector to an artificial receptor, which is expressed on the surface of 293/6H cells (FIG. 3). The extracellular domain of this artificial receptor (AR) is an anti-5His (SEQ ID NO: 19) single chain antibody, which is genetically fused with the transmembrane domain of the platelet derived growth factor receptor (Douglas et al., Nat Biotechnol 17, 470-5 (1999)). In addition to receptor binding, this 6His (SEQ ID NO: 17) sequence was employed to facilitate the detection and purification of the FF/6H chimeras and Ad virions incorporating this protein.

In the adenovirus of the present invention, the targeting ligand is a single chain antibody, preferably a scFv ligand, more preferably a stabilized scFv. In a preferred embodiment of the invention, the stabilized scFv ligand comprises mutations in the scFv CDR regions. Any mutations which preserve an ability of scFv in the context of Ad capsid binds an antigen are suitable for methods of the invention. Examples of scFv stabilizing mutations include, but are not limited to, those mutations described in Arndt et al., J Mol Biol 2001 Sep. 7;312(1):221-8; Bestagno et al., Biochemistry 2001 Sep. 4;40(35): 10686-92 and Rajpal et al., Proteins 2000 Jul. 1;40(1):49-57, the disclosures of which are incorporated by reference. A stabilized scFv “framework” is developed via directed mutations in the scFv CDR regions. These stabilized CDRs' framework can then serve as a scaffod onto which scFv variable domains, which embody antigen recognition, can then be grafted by molecular engineering methods. The chimeric scFv thus manifests the desired antigen recognition profile while also embodying the stability of the scaffold CDR domain. Other methods for scFv stabilization may also be used in the methods of the present invention.

In a preferred embodiment, the stabilized scFv ligand is targeted to a cell surface marker of a tumor cell. Cell surface markers that can be targeted according to the methods of the present invention include, but are not limited to, CD40, DC-SIGN, DEC-205, CEA and PSMA. In one embodiment, the stabilized scFv ligand is an anti-CD40 scFv.

In one embodiment, the adenovirus carries in its genome a transgene, which can be therapeutic gene. A representative example of a therapeutic gene is a herpes simplex virus thymidine kinase gene. Other target transgenes include, but are not limited to, cytosine deaminase (CD) and a fusion of cytosine deaminase and uracilphosphoribosyltransferase (CD/UPRT).

In another embodiment, the invention encompasses a method for inhibiting tumor cell growth in a subject in need thereof comprising administering to the subject in need thereof a therapeutically effective amount of the adenovirus described herein wherein the scFv ligand targets the tumor cell such that the adenovirus infects the tumor cells and thereby inhibits tumor cell growth in the subject. In one embodiment, the adenovirus further comprises a transgene. In an embodiment wherein the transgene is herpes simplex virus thymidine kinase the method for inhibiting tumor cell growth can optionally comprise administering ganciclovir. Another agent that can be co administered in combination with a transgene is 5-fluorocytosine (5FC).

In accordance with the present invention, there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984). Therefore, if appearing herein, the following terms shall have the terminology set out below.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure herein according to the normal convention of giving only the sequence in the 5′ to 3′ direction along the nontranscribed strand of DNA (i.e., the strand having a sequence homologous to the mRNA).

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo; i.e., capable of replication under its own control. An “origin of replication” refers to those DNA sequences that participate in DNA synthesis. An “expression control sequence” is a DNA sequence that controls and regulates the transcription and translation of another DNA sequence. A coding sequence is “operably linked” and “under the control” of transcriptional and translational control sequences in a cell when RNA polymerase transcribes the coding sequence into mRNA, which is then translated into the protein encoded by the coding sequence.

In general, expression vectors containing promoter sequences which facilitate the efficient transcription and translation of the inserted DNA fragment are used in connection with the host. The expression vector typically contains an origin of replication, promoter(s), terminator(s), as well as specific genes which are capable of providing phenotypic selection in transformed cells. The transformed hosts can be fermented and cultured according to means known in the art to achieve optimal cell growth.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence. A “cDNA” is defined as copy-DNA or complementary-DNA, and is a product of a reverse transcription reaction from an mRNA transcript.

Transcriptional and translational control sequences are DNA regulatory sequences, such as promoters, enhancers, polyadenylation signals, terminators, and the like, that provide for the expression of a coding sequence in a host cell. A “cis-element” is a nucleotide sequence, also termed a “consensus sequence” or “motif”, that interacts with other proteins which can upregulate or downregulate expression of a specific gene locus. A “signal sequence” can also be included with the coding sequence. This sequence encodes a signal peptide, N-terminal to the polypeptide, that communicates to the host cell and directs the polypeptide to the appropriate cellular location. Signal sequences can be found associated with a variety of proteins native to prokaryotes and eukaryotes.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes. Prokaryotic promoters contain Shine-Dalgarno sequences in addition to the −10 and −35 consensus sequences.

The term “oligonucleotide” is defined as a molecule comprised of two or more deoxyribonucleotides, preferably more than three. Its exact size will depend upon many factors which, in turn, depend upon the ultimate function and use of the oligonucleotide. The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use for the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence to hybridize therewith and thereby form the template for the synthesis of the extension product.

As used herein, the terms “restriction endonucleases” and “restriction enzymes” refer to enzymes which cut double-stranded DNA at or near a specific nucleotide sequence.

“Recombinant DNA technology” refers to techniques for uniting two heterologous DNA molecules, usually as a result of in vitro ligation of DNAs from different organisms. Recombinant DNA molecules are commonly produced by experiments in genetic engineering. Synonymous terms include “gene splicing”, “molecular cloning” and “genetic engineering”. The product of these manipulations results in a “recombinant” or “recombinant molecule”.

A cell has been “transformed” or “transfected” with exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a vector or plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary cell that is capable of stable growth in vitro for many generations. An organism, such as a plant or animal, that has been transformed with exogenous DNA is termed “transgenic”.

As used herein, the term “host” is meant to include not only prokaryotes but also eukaryotes such as yeast, plant and animal cells. Prokaryotic hosts may include E. coli, S. tymphimurium, Serratia marcescens and Bacillus subtilis. Eukaryotic hosts include yeasts such as Pichia pastoris, mammalian cells and insect cells and plant cells, such as Arabidopsis thaliana and Tobaccum nicotiana.

Two DNA sequences are “substantially homologous” when at least about 75% (preferably at least about 80%, and most preferably at least about 90% or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. In another example, the coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

In addition, the invention may includes portions or fragments of the fiber or fibritin genes. As used herein, “fragment” or “portion” as applied to a gene or a polypeptide, will ordinarily be at least 10 residues, more typically at least 20 residues, and preferably at least 30 (e.g., 50) residues in length, but less than the entire, intact sequence. Fragments of these genes can be generated by methods known to those skilled in the art, e.g., by restriction digestion of naturally occurring or recombinant fiber or fibritin genes, by recombinant DNA techniques using a vector that encodes a defined fragment of the fiber or fibritin gene, or by chemical synthesis.

As used herein, “chimera” or “chimeric” refers to a single transcription unit possessing multiple components, often but not necessarily from different organisms. As used herein, “chimeric” is used to refer to tandemly arranged coding sequence (in this case, that which usually codes for the adenovirus fiber gene) that have been genetically engineered to result in a protein possessing region corresponding to the functions or activities of the individual coding sequences.

The “native biosynthesis profile” of the chimeric fiber protein as used herein is defined as exhibiting correct trimerization, proper association with the adenovirus capsid, ability of the ligand to bind its target, etc. The ability of a candidate chimeric fiber-fibritin-ligand protein fragment to exhibit the “native biosynthesis profile” can be assessed by methods described herein.

A standard Northern blot assay can be used to ascertain the relative amounts of mRNA in a cell or tissue in accordance with conventional Northern hybridization techniques known to those persons of ordinary skill in the art. Alternatively, a standard Southern blot assay may be used to confirm the presence and the copy number of the gene of interest in accordance with conventional Southern hybridization techniques known to those of ordinary skill in the art. Both the Northern blot and Southern blot use a hybridization probe, e.g. radiolabelled cDNA or oligonucleotide of at least 20 (preferably at least 30, more preferably at least 50, and most preferably at least 100 consecutive nucleotides in length). The DNA hybridization probe can be labelled by any of the many different methods known to those skilled in this art.

Hybridization reactions can be performed under conditions of different “stringency.” Conditions that increase stringency of a hybridization reaction are well known. See for examples, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The labels most commonly employed for these studies are radioactive elements, enzymes, chemicals which fluoresce when exposed to untraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

As used herein, the terms “fiber gene” and “fiber” refer to the gene encoding the adenovirus fiber protein. As used herein, “chimeric fiber protein” refers to a modified fiber gene as described above.

As used herein the term “physiologic ligand” refers to a ligand for a cell surface receptor.

The present invention is directed to a vector system that provides both a highly efficient and specific targeting of adenovirus vector for the purpose of in vivo gene delivery to predefined cell types after administration. In the recombinant adenoviral vector of the present invention, a fiber replacement protein comprising a fiber-fibritin-ligand is employed to target adenoviral vector to a specific cell for gene therapy. This is accomplished by the construction of adenoviral vectors which contain fiber-fibritin-ligand chimeras. These adenoviral vectors are capable of delivering gene products with high efficiency and specificity to cells expressing receptors which recognize the ligand component of the fiber-fibritin-ligand chimera. A person having ordinary skill in this art would recognize that one may exploit a wide variety of genes encoding e.g. receptor ligands or antibody fragments which specifically recognize cell surface proteins unique to a particular cell type to be targeted.

The invention also encompasses viral vectors, preferably an adenoviral vector comprising the adenovirus of described herein. In one embodiment, adenovirus is operatively linked to a non-viral promoter.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996;93:11313-11318; Ballay et al., EMBO J. 1993;4:3861-65; Felgner et al., J. Biol. Chem. 1994;269:2550-2561; Frolov et al., Proc. Natl. Acad. Sci. USA 1996;93:11371-11377; Graham, Tibtech 1990;8:85-87; Grunhaus et al., Sem. Virol. 1992;3:237-52; Ju et al., Diabetologia 1998;41:736-739; Kitson et al., J. Virol. 1991;65:3068-3075; McClements et al., Proc. Natl. Acad. Sci. USA 1996;93:11414-11420; Moss, Proc. Natl. Acad. Sci. USA 1996;93:11341-11348; Paoletti, Proc. Natl. Acad. Sci. USA 1996;93:11349-11353; Pennock et al., Mol. Cell. Biol. 1984;4:399-406; Richardson (Ed), Methods in Molecular Biology 1995;39, “Baculovirus Expression Protocols,” Humana Press Inc.; Smith et al. (1983) Mol. Cell. Biol. 1983;3:2156-2165; Robertson et al., Proc. Natl. Acad. Sci. USA 1996;93:11334-11340; Robinson et al., Sem. Immunol. 1997;9:271; and Roizman, Proc. Natl. Acad. Sci. USA 1996;93:11307-11312.

According to one embodiment of the invention, the expression vector is a viral vector, in particular an in vivo expression vector. In an advantageous embodiment, the expression vector is an adenovirus vector, such as a human adenovirus (HAV) or a canine adenovirus (CAV). Advantageously, the adenovirus is a human Ad5 vector, an E1-deleted adenovirus or an E3-deleted adenovirus.

In one embodiment the viral vector is a human adenovirus, in particular a serotype 5 adenovirus, rendered incompetent for replication by a deletion in the E1 region of the viral genome. The deleted adenovirus is propagated in E1-expressing 293 cells or PER cells, in particular PER.C6 (F. Falloux et al Human Gene Therapy 1998, 9, 1909-1917). The human adenovirus can be deleted in the E3 region eventually in combination with a deletion in the E1 region (see, e.g. J. Shriver et al. Nature, 2002, 415, 331-335, F. Graham et al Methods in Molecular Biology Vol. 7: Gene Transfer and Expression Protocols Edited by E. Murray, The Human Press Inc, 1991, p 109-128; Y. Ilan et al Proc. Natl. Acad. Sci. 1997, 94, 2587-2592; S. Tripathy et al Proc. Natl. Acad. Sci. 1994, 91, 11557-11561; B. Tapnell Adv. Drug Deliv. Rev. 1993, 12, 185-199; X. Danthinne et al Gene Thrapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter). The CMV-IE promoter is advantageously of murine or human origin. The promoter of the elongation factor 1α can also be used. In one particular embodiment a promoter regulated by hypoxia, e.g. the promoter HRE described in K. Boast et al Human Gene Therapy 1999, 13, 2197-2208), can be used. A muscle specific promoter can also be used (X. Li et al Nat. Biotechnol. 1999, 17, 241-245). Strong promoters are also discussed herein in relation to plasmid vectors. A poly(A) sequence and terminator sequence can be inserted downstream the polynucleotide to be expressed, e.g. a bovine growth hormone gene or a rabbit β-globin gene polyadenylation signal.

In another embodiment the viral vector is a canine adenovirus, in particular a CAV-2 (see, e.g. L. Fischer et al. Vaccine, 2002, 20, 3485-3497; U.S. Pat. No. 5,529,780; U.S. Pat. No. 5,688,920; PCT Application No. WO95/14102). For CAV, the insertion sites can be in the E3 region and/or in the region located between the E4 region and the right ITR region (see U.S. Pat. No. 6,090,393; U.S. Pat. No. 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit β-globin gene polyadenylation signal.

The invention also provides for transformed host cells comprising such vectors. In one embodiment, the vector is introduced into the cell by transfection, electroporation or transformation. The invention also provides for a method for preparing a transformed cell expressing the adenovirus of the present invention comprising transfecting, electroporating or transforming a cell with the adenovirus to produce a transformed host cell and maintaining the transformed host cell under biological conditions sufficient for expression of the adenovirus in the host cell.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

It is understood to one of skill in the art that conditions for culturing a host cell varies according to the particular gene and that routine experimentation is necessary at times to determine the optimal conditions for culturing the vector depending on the host cell. A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

A “fiber replacement protein” is a protein that substitutes for fiber and provide 3 essential feature: trimerizes like fiber, lacks adenoviral tropism and has novel tropism.

As used herein, “chimera” or “chimeric” refers to a single polypeptide possessing multiple components, often but not necessarily from different organisms. As used herein, “chimeric” is used to refer to tandemly arranged protein moieties that have been genetically engineered to result in a fusion protein possessing regions corresponding to the functions or activities of the individual protein moieties.

As used herein, the terms “fiber gene” refer to the gene encoding the adenovirus fiber protein. As used herein, “chimeric fiber protein” refers to a modified fiber as defined above.

A “fiber replacement protein” is a protein that substitutes for fiber and provide three essential features: trimerizes like fiber, lacks adenoviral tropism and has novel tropism.

As used herein the term “physiologic ligand” refers to a ligand for a cell surface receptor.

In addition, the invention may includes portions or fragments of the fiber or fibritin proteins. As used herein, “fragment” or “portion” as applied to a protein or a polypeptide, will ordinarily be at least 10 residues, more typically at least 20 residues, and preferably at least 30 (e.g., 50) residues in length, but less than the entire, intact sequence. Fragments of these genes can be generated by methods known to those skilled in the art, e.g., by restriction digestion of naturally occurring or recombinant fiber or fibritin genes, by recombinant DNA techniques using a vector that encodes a defined fragment of the fiber or fibritin gene, or by chemical synthesis.

The following examples are given for the purpose of illustrating various embodiments of the invention and are not meant to limit the present invention in any fashion.

### EXAMPLES

### Example 1

### Construction of the Fiber-Fibritin-6His (SEQ ID NO: 13) (FF/6H) Chimera

Generation of the gene encoding the fiber-fibritin-6His chimera was done in several steps. First, a segment of the fibritin gene was PCR-amplified and used to substitute most of the fiber gene sequence encoding the shaft domain. For this, a portion of the T4 fibritin gene encoding the sixth coiled coil through the C-terminal of the protein was amplified with a pair of primers “FF.F” (GGG AAC TTG ACC TCA CAG AAC GTT TAT AGT CGT TTA AAT G) (SEQ ID NO. 1) and “FF.R” (AGG CCA TGG CCA ATT TTT GCC GGC GAT AAA AAG GTA G) (SEQ ID NO. 2). The product of this PCR encodes a segment of an open reading frame (ORF) containing four amino terminal (GLNT) (SEQ ID NO: 20) and three carboxy terminal (KIG) codons of the fiber shaft sequence fused to the fibritin sequence. The reverse primer introduces a silent mutation at the 3′ end of the fibritin open reading frame resulting in generation of a unique NaeI-site. Also, NcoI-site was incorporated in the “FF.F” in order to fuse the open reading frame of the fiber and the fibritin. The product of the PCR was then cleaved with NcoI and cloned in the fiber shuttle vector pNEB.PK3.6 (Krasnykh et al., J. Virol. 70:6839-46 (1996)) cut with NaeI and NcoI. As a result of this cloning, an original NaeI-site in the fiber open reading frame was destroyed, therefore NaeI-site at the end of the fibritin open reading frame remains unique. The plasmid generated was named pNEB.PK.FF_{BB}. This fusion procedure resulted in an open reading frame, in which the fiber and the fibritin sequence were joined via an SQNV peptide (SEQ ID NO: 18) hinge, present at the beginning of the 3rd repeat of Ad fiber shaft as well as at the 6th coil coiled segment of the fibritin.

At the next step, a portion of 3′ terminal sequence of FF. open reading frame was replaced with synthetic oligo duplex in order to introduce in the construct a unique restriction site, SwaI, which would allow modifications of the 3′ end of the gene. To reach this end, a duplex made of oligos “F5. Δ3Swa.T” (TTG GOC CCA TTT AAA TGA ATC GTT TGT GTT ATG TTT CAA CGT GTT TAT TTT TC) (SEQ ID NO. 3) and “F5. Δ3.Swa.B” (AAT TGA AAA ATA AAC ACG TTG AAA CAT AAC ACA AAC GAT TCA TTT AAA TGG GGC CAA TAT T) (SEQ ID NO. 4) was cloned in BstXI-MfeI-digested pNEB.PK3.6, thereby generating pNEB.PK Δ3.

To facilitate the downstream manipulation with the 3′ end of the fiber-fibritin gene a plasmid pNEB.PK.FF_{BB}Δ3 was generated as follows: an NcoI-Acc65.1-fragment in pNEB.PK.FF_{BB} was replaced with an NcoI-Acc65.I-fragment from pNEB.PKΔ3.

The plasmid pXK.FF_{BB}Δ3 was obtained from pNEB.PK.FF_{BB}Δ3 by deleting a XbaI-fragment containing a portion of the Ad5 Luc-3 DNA. This was done in order to eliminate a BamHI site contained in this XbaI fragment, which would otherwise compromise the utility of the BamHI-site introduced into the construct at a later step (see below).

To add the sequence encoding a C-terminal linker to the fiber/fibritin fusion protein, a synthetic oligo duplex consisting of oligos “FF_{BB}LL.T” (GGC AGG TGG AGG CGG TTC AGG CGG AGG TGG CM TGG OGG TGG OGG ATC OGG GGA TTT) (SEQ ID NO. 5) and “FF_{BB}LL.B” (AAA TCC COG GAT COG CCA CGG CCA GAG CCA CCT COG CCT GAA CM CCTCCACCTGCC) (SEQ ID NO. 6) was cloned into NaeI-SwaI-digested pXK.FF_{BB}Δ3, generating pXK.FF_{BB}LL. The duplex contains a BamHI-site at the 3′-end of the linker-encoding sequence. Of note, this cloning procedure left both the NaeI- and the SwaI-sites intact and, therefore available for subsequent cloning steps.

An RGS (His)6-encoding sequence (SEQ ID NO: 16) was fused to the 3′ end of the FF_{BB}LL gene by inserting a synthetic oligo duplex made of oligos “RGS6H.T” (GAT CTA GAG GAT CGC ATC ACC ATC ACC ATC ACT AAT) (SEQ ID NO. 7) and “RGS6H.B” (ATT AGT GAT GGT GAT GGT GAT GCG ATC CTC TA) (SEQ ID NO. 8) into BamHI-SwaI-digested pXK.FF_{BB}LL. The resultant plasmid was designated pXK.FF/6H. This cloning procedure destroyed both the BamHI- and the SwaI-sites. This completed the derivation of the shuttle plasmid containing the FF/6H gene.

In order to express the FF/6H protein in E. coli, the FF/6H assembled in pXK.FF/6H was PCR amplified using the primers “FF.F(BspHI) (CCC TCA TGA AGC GCG CAA GAC CGT CTG) (SEQ ID NO. 9) and (CCC AAG CTT AGT GAT GGT GAT GGT GAT) (SEQ ID NO. 10), digested with NcoI and HindIII and cloned into NcoI-HindIII-cut pQE60 resulting in pQE.FF/6H.

In order to derive recombinant adenoviral genome containing FF/6H gene, an EcoRI-XbaI-fragment of pXK.FF/6H was used for recombination with SwaI-digested pVK500 (Dmitriev et al., J Virol 72, 9706-13 (1998)), resulting in pVK511. The luciferase expressing cassette was then incorporated in place of the E1 region of the adenoviral genome contained in pVK511 via homologous DNA recombination between ClaI-digested pVK511 and a fragment of pACCMV.LucΔPC. The plasmid generated was designated pVK711. The virus of interest, Ad5LucFF/6H, was then rescued by transfecting 211B cells (Von Seggern et al., J Gen Virol 79, 1461-8 (1998)) with PacI-digested pVK711.

### Example 2

### Characterization of Recombinant Adenovirus Expressing the Fiberfibritin-6His (SEQ ID NO: 13)(FF/6H) Chimera

For the purposes of preliminary characterization, the FF/6H chimeric protein was initially expressed in E. coli and purified on a Ni-NTA-agarose column. Subsequent SDS-PAGE analysis of the purified chimeric protein proved that it is trimeric and that the FF/6H trimers are as stable in an SDS-containing gel as the trimers of the wild type Ad5 fiber (FIG. 1B). Efficient binding of the FF/6H protein to a Ni-NTA-containing matrix proved that the 6His ligand (SEQ ID NO: 17) was available for binding in the context of this trimeric molecule. According to this analysis, truncated T4 fibritin incorporated into the FF/6H protein was able to direct trimerization of the chimera and also successfully served the purposes of ligand presentation, thereby satisfying two key functional criteria of an ideal fiber-replacing molecule.

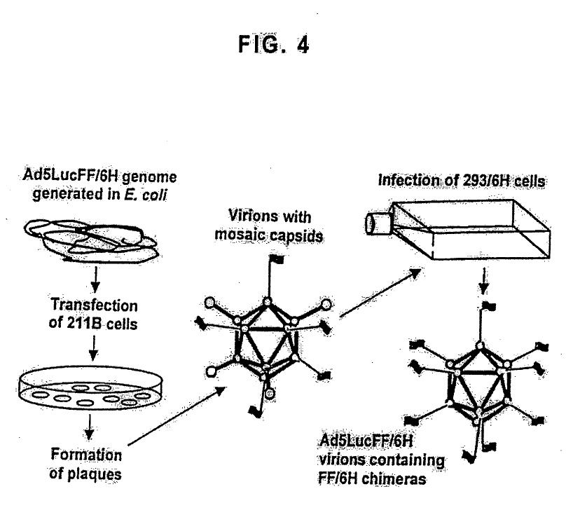

In order to evaluate the functional utility of the FF/6H chimeras incorporated into a mature adenoviral particle, homologous recombination in E. coli (Krasnykh et al., J Virol 72, 1844-52 (1998)) was employed to insert the FF/6H encoding gene into the genome of E1-deleted, firefly luciferase expressing Ad5 in place of the wild type fiber gene. The virus of interest, Ad5LucFF/6H, was then rescued by transfection of 211B cells with the resultant adenoviral genome (FIG. 4). 211B cells, a derivative of 293 cells which constitutively express the wild type Ad5 fiber protein (Von Seggern et al., J Gen Virol 79, 1461-8 (1998)), were chosen for this transfection experiment in order to guarantee the success of the virus rescue. Ad5LucFF/6H was further expanded on 211B cells and purified by double banding in a CsCl gradient. At this point, the viral stock contained mosaic virions bearing a mixture of the wild type fibers and FF/6H chimeras (data not shown). In order to obtain a homogenous population of Ad5LucFF/6H virions lacking the wild type fibers, but exclusively incorporating FF/6H proteins, the original viral stock was then used to infect 293/6H cells at multiplicity of infection of 1000 viral particles per cell. CsCl gradient purification of Ad5LucFF/6H virions isolated from the lysates of infected 293/6H cells 72 hours post infection (at which point a complete cytopathic effect was observed) resulted in a yield of 3×10⁴ viral particles per cell, which was well within the range of yields characteristic for E1-deleted Ad5 vectors.

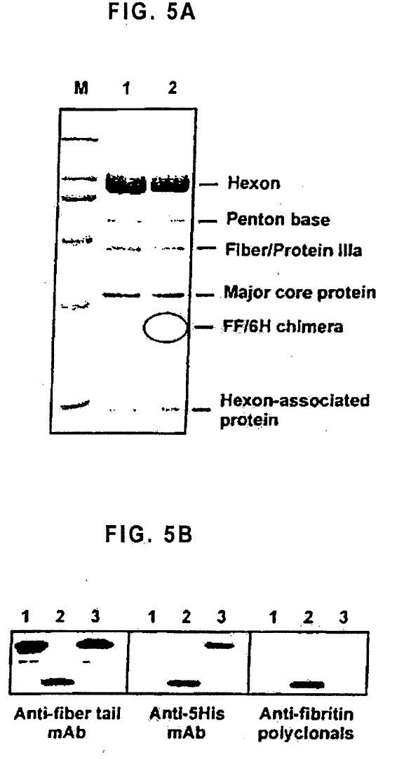

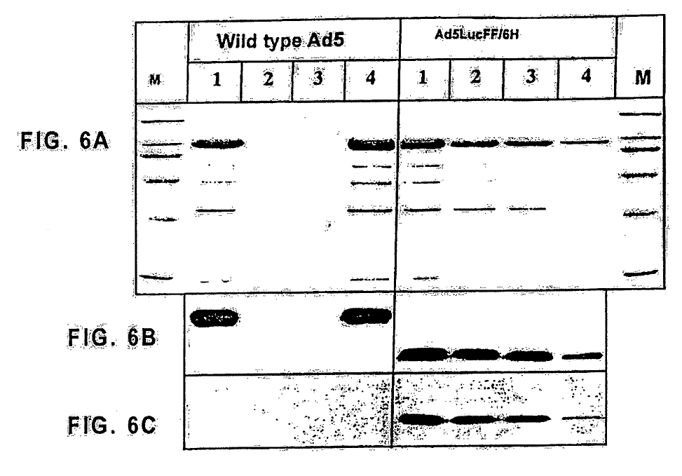

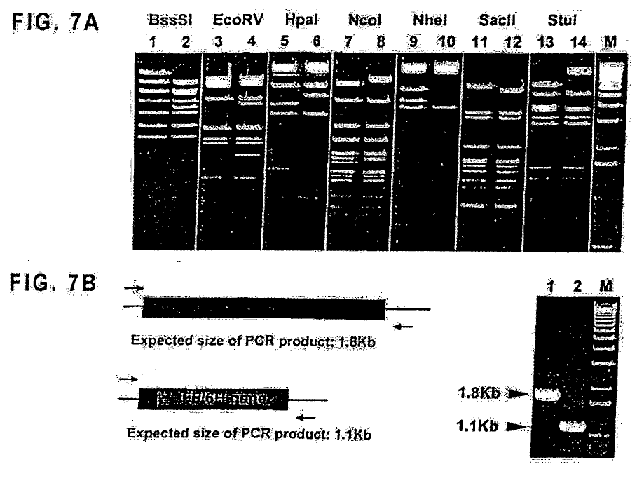

The next goal was to demonstrate that the FF/6H chimeras had been incorporated into the Ad5LucFF/6H capsids. Since fiberless Ad5 virions have been successfully purified on CsCl gradients by others (Von Seggern et al., J Gen Virol 79, 1461-8 (1998) and Legrand et al., J Virol 73, 907-19 (1999)), it was possible that the putative Ad5LucFF/6H virions isolated in our study could have lacked FF/6H proteins. This was ruled out by SDS-PAGE of purified Ad5LucFF/6H virions and a Western blot analysis utilizing anti-sera specific to all three major components of FF/6H chimera, the fiber tail, the fibritin and the 6His ligand (SEQ ID NO: 17) (FIGS. 5A and B). These assays showed that the capsid of Ad5LucFF/6H virions consists of completely matured Ad proteins and incorporates full-size FF/6H chimeras. As expected, no wild type fibers were found in this preparation of Ad5LucFF/6H. These findings were further corroborated in an experiment involving binding of purified Ad5LucFF/6H virions to Ni-NTA-resin: in contrast to the Ad vector containing wild type fibers, which did not bind to the matrix, Ad5LucFF/6H demonstrated 6His-mediated (SEQ ID NO: 17) binding to the resin (FIG. 6). Therefore, in addition to its ability to assume a trimeric configuration and bind to a receptor-mimicking molecule, the FF/6H chimera also retained the capacity of being incorporated into mature Ad capsids.

Restriction enzyme analysis of the Ad5LucFF/6H genome, diagnostic PCR utilizing a pair of primers flanking the fiber gene in Ad5 genome and partial sequencing of Ad5LucFF/6H DNA demonstrated that the viral genome was stable and that the only fiber-encoding gene present was the FF/6H gene (FIG. 7). This set of experiments completed the molecular characterization of Ad5LucFF/6H by confirming both the identity and the integrity of the virus capsid and its genome.

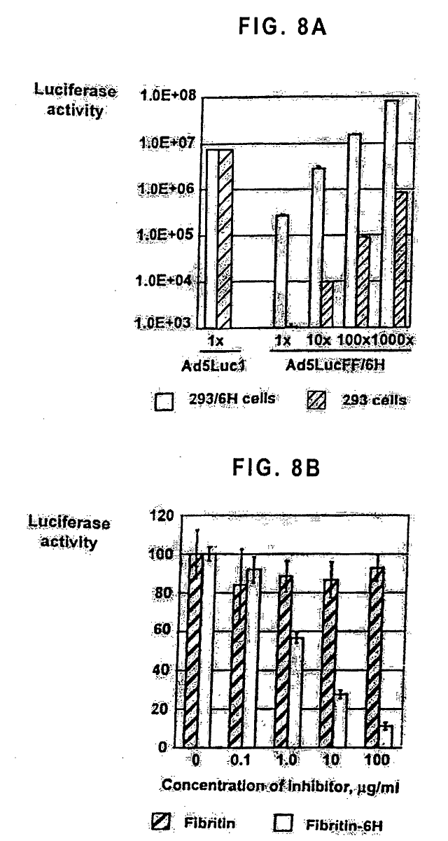

The ability of Ad5LucFF/6H to deliver a transgene to the target cells was then evaluated in a series of studies employing this viral vector for infection of 293/6H cells expressing an artificial receptor capable of binding proteins and Ad virions possessing a 6His tag (SEQ ID NO: 17) (FIG. 3). First, the gene transfer capacity of Ad5LucFF/6H was compared to that of an isogenic Ad vector, Ad5Luc1, bearing wild type fibers (FIG. 8A). The doses of both viruses used in this experiment were normalized based on the particle titers of the viral preparations, which also correlated well with the total protein concentration of the samples. Due to the significant differences in the dissociation constants (k_{d}) previously determined for the Ad5 fiber/CAR interaction (Davison et al., J Virol 73, 4513-4517 (1999)), 4×10⁻⁹ M, and for the 5 His/anti-5His (SEQ ID NO: 19) mA b 3D5 interaction (Lindner et al., Biotechniques 22, 140-9 (1997)), 4.75×10⁻⁷ M, lower efficiency of the gene transfer for Ad5LucFF/6H vector was expected.

In order to compensate for potentially lower infection levels resulting from this difference in binding affinities, several different doses of Ad5LucFF/6H vector were used, of which the lowest corresponded to the dose of the control vector. This experiment showed that Ad5LucFF/6H was capable of efficient transgene delivery to the target cells. However, at equal multiplicities of infection the level of transgene expression in Ad5Luc1-infected cells (293 and 293/6H) was 30-fold higher than that registered in 293/6H cells infected with Ad5LucFF/6H. Importantly, there was a two order of magnitude increase in Ad5LucFF/6H-expressed luciferase activities detected in 293/6H cells expressing AR compared to parental 293 cells infected with the same vector. This differential in the transgene expression levels strongly suggests that Ad5LucFF/6H-mediated gene transfer to 293/6H occurred in a CAR-independent, receptor-specific manner via interaction of the virus with the AR.

The next gene transfer experiment employed two different forms of recombinant fibritin proteins as blocking agents, of which only one, fibritin-6H, contained a carboxy terminal 6His tag (SEQ ID NO: 17) (FIG. 8B). The purpose of this assay was to provide additional evidence that the backbone of the fibritin molecule does not contribute to binding to AR or any other cell surface receptor. Dose-dependent inhibition of Ad5LucFF/6H infection of 293/6H cells with fibritin-6H, but not with the fibritin lacking the 6His tag (SEQ ID NO: 17), further proved that this tag is the component of the virion solely responsible for the binding of the virus to the AR.

The present invention has developed a novel approach to the modification of adenoviral vector tropism by replacing the receptor-binding fiber protein in the adenoviral capsid with an artificial protein chimera. The rational design of this chimera, based on the general structural similarity of the Ad5 fiber and bacteriophage T4 fibritin, has resulted in the derivation of a novel ligand-presenting molecule. The most important difference from the wild type fiber protein is the disengagement of the trimerization and the receptor-binding functions normally performed by the fiber knob domain. As a result of this distribution of functions, the receptor specificity of the re-engineered Ad5 vector may now be defined by a domain of the chimera which plays no role in the trimerization of the molecule, and may therefore be manipulated without the risk of destabilizing the ligand-presenting protein and the virion. The use of T4 fibritin for ligand display suggests that a wide variety of heterologous targeting ligands, including large polypeptide molecules, may be employed in the context of the fiberfibritin chimera described here.

Fibritin chimeras analogous to the one described in this work may be viewed as versatile ligand-displaying molecules suitable for genetic modification of virtually any human or animal adenoviral vector. The problem of elimination of undesirable natural tropism of native fibers contained in the adenoviral virion may thus be solved by substitution of native fibers with such fibritin chimeras. This approach has significant advantage over maneuvers involving the identification and subsequent mutagenesis of the native receptor binding sites within the fibers of numerous adenoviral species, some of which are able to bind to different types of primary receptors. In addition, this strategy eliminates the risk of reversion of the mutated fiber gene to the wild type during multiple rounds of propagation, which would compromise the efficiency of any vector targeting schema.

An additional advantage offered by adenoviral vectors incorporating the fibritin-based chimeras for the purposes of human gene therapy because of interference of anti-fiber antibodies present in the serum of some gene therapy patients with the adenoviral vectors used in clinical protocols. Importantly, these antibodies have been shown to have a synergistic effect on adenoviral vector neutralization when present together with anti-penton base antibodies. Thus, deletion of the most of the fiber sequence in the fibritin-bearing adenoviral vectors would make them refractory to this type of immune response and therefore more efficient a s therapeutic agents.

### Example 3

### Characterization of Recombinant Adenovirus Expressing the Fiberfibritin-RGD-6His (SEQ ID NO: 13) (FF.RGD/6H) Chimera

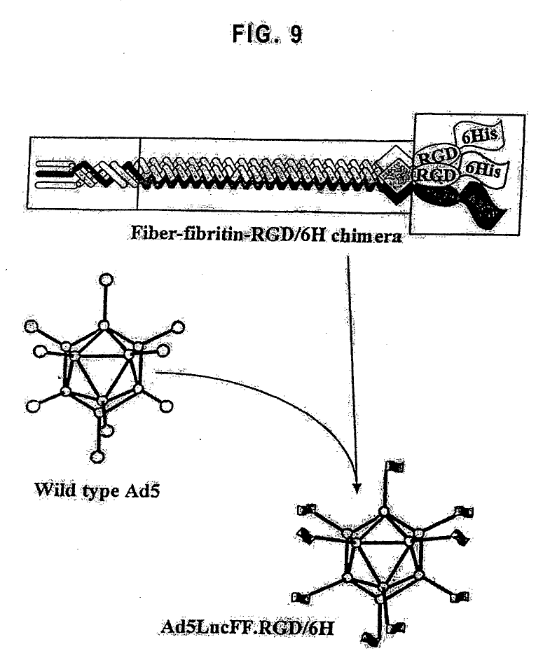

A second adenoviral vector, Ad5luc.FF.RGD/6H, containing fiber-fibritin chimeras incorporating at their carboxy termini two peptide ligands RGD-4C (CDCRGDCFC) (SEQ ID NO. 14) and 6His (SEQ ID NO: 17) was generated (FIG. 9). The virus was propagated in 293 cells and purified on CsCl gradient according to standard technique.

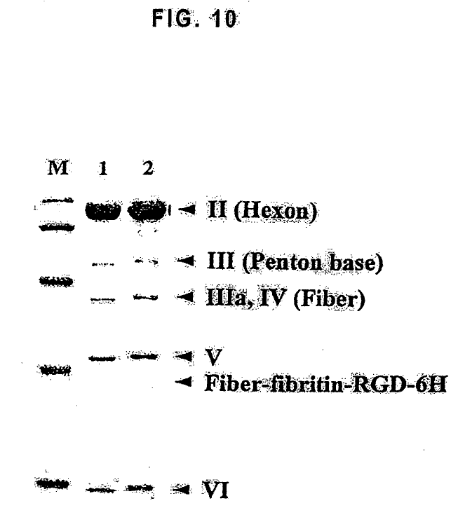

The protein composition of Ad5luc.FF.RGD/6H was verified by SDS-PAGE using the virus with wild type capsids as a control. As shown in FIG. 10, all major protein components of Ad5luc.FF.RGD/6H are essentially the same as those of control adenoviral capsid. The only difference noted between the capsid protein patters demonstrated by the two viruses was the presence of the FF.RGD/6H chimeras in the Ad5LucFF.RGD/6H particles in place of the wild type fibers contained in the capsids of the control adenovirus.

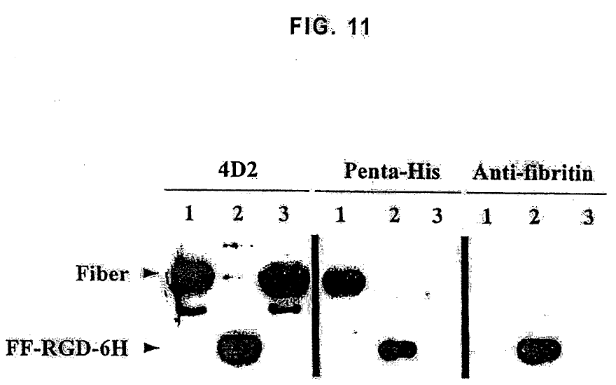

FF.RGD/6H chimeras present in the preparation of Ad5luc.FF.RGD/6H were further identified by Western blot analysis utilizing a set of antibodies specific to each of the component of the chimeric protein. The presence of the fiber tail domain, the fibritin fragment and the 6His tag (SEQ ID NO: 17) was confirmed by using relevant mono- and polyclonal antibodies (FIG. 11).

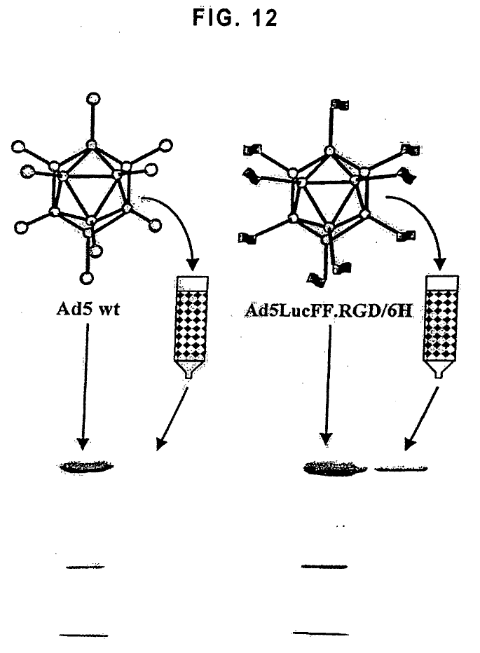

Association of the FF.RGD/6H chimeras with the Ad5luc.FF.RGD/6H particles was proved by incubating purified Ad5luc.FF.RGD/6H virions with Ni-NTA-sepharose which is designed for purification of the 6His-tagged (SEQ ID NO: 17) proteins. In contrast to control adenoviral vector containing wild type fibers which did not bind to Ni-NTA, Ad5luc.FF.RGD/6H was efficiently retained on the column. The presence of all major adenoviral capsid proteins in the material eluted from the resin with imidazole suggested that the Ad5luc.FF.RGD/6H virions were anchored to Ni-NTA-sepharose by virtue of the 6His-containing (SEQ ID NO: 17) fiber-fibritin chimeras associated with the virions (FIG. 12).

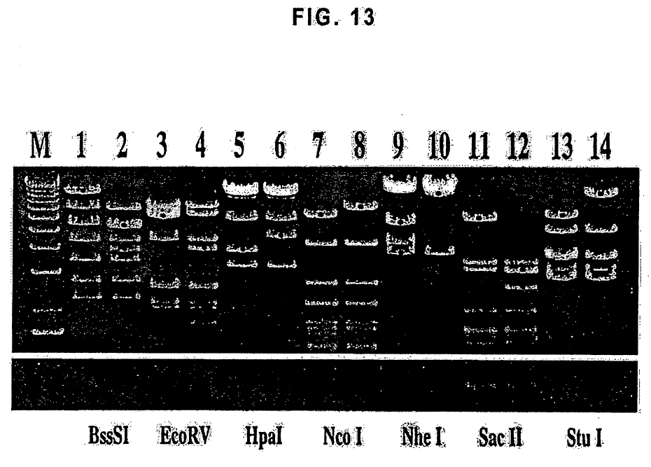

In order to rule out the possibility of contamination of Ad5luc.FF.RGD/6H preparation with another adenoviral vector, Ad5luc.FF.RGD/6H DNA isolated from virions was subjected to three different assay including restriction enzyme analysis (FIG. 13), “diagnostic” PCR, and sequencing of the fiber-fibritin gene as well as the regions of Ad genome adjacent to it. All three assays showed that the preparation of Ad5luc.FF.RGD/6H is free from any contaminating adenovirus and therefore is suitable for subsequent studies aimed to characterize the gene transfer capacity and the cell entry pathway utilized by Ad5luc.FF.RGD/6H.

To evaluate the gene transfer capacity of Ad5luc.FF.RGD/6H, the virus was employed for gene delivery experiments utilizing two different cell lines: 293 and 293/6H. The latter of the two lines is the derivative of 293 cells constitutively expressing artificial receptor capable of binding 6His-tagged (SEQ ID NO: 17) proteins. The luciferase-expressing adenoviral vector isogenic to Ad5luc.FF.RGD/6H but incorporating the wild type fibers was used in these experiments as a control. The gene transfer with the control virus was done at one multiplicity of infection (MOI), whereas Ad5luc.FF.RGD/6H was used at different MOIs.

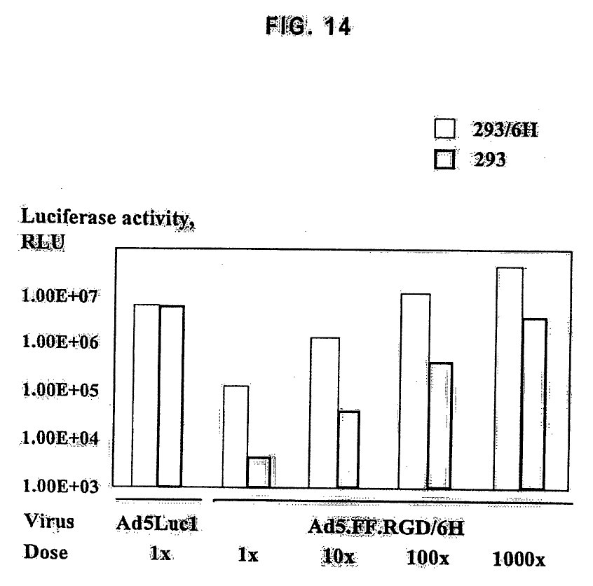

As shown in FIG. 14, Ad5luc.FF.RGD/6H can deliver a luciferase reporter to both types of cells, although with rather different efficiencies (luciferase expression in naive 293 cells was always lower than in 293/6H cells), thereby suggesting that both the RGD-4C (SEQ ID NO: 14) and the 6His peptides (SEQ ID NO: 17) incorporated within the FF.RGD/6H chimeras functioned as targeting ligands.

### Example 4

### Genetically Targeted Adenovirus Vector Directed to CD40-Expressing Cells

Applicants described the use of an adenovirus (Ad) fiber replacement strategy for genetic targeting of the virus to human CD40, which is expressed by a variety of diseased tissues (see Belousova et al., J. Virol. 2003 November;77(21):11367-77, the disclosure of which is incorporated by reference in its entirety). The tropism of the virus was modified by the incorporation into its capsid of a protein chimera comprising structural domains of three different proteins: the Ad serotype 5 fiber, phage T4 fibritin, and the human CD40 ligand (CD40L). The tumor necrosis factor-like domain of CD40L retains its functional tertiary structure upon incorporation into this chimera and allows the virus to use CD40 as a surrogate receptor for cell entry. The ability of the modified Ad vector to infect CD40-positive dendritic cells and tumor cells with a high efficiency makes this virus a prototype of choice for the derivation of therapeutic vectors for the genetic immunization and targeted destruction of tumors.

Applicant demonstrated the versatility of this fiber replacement strategy by creating an Ad vector targeted to human CD40 by virtue of the incorporation of the CD40 ligand (CD40L) into its capsid. The study showed that despite the significant size of the ligand used and its complex tertiary structure, both components of the targeting protein, the CD40L domain and the FF backbone, folded properly, thereby making the entire chimera fully functional. Importantly, for the first time, a pair of cell surface molecules which are normally involved in an intercellular interaction was used as a component of an alternative cell entry pathway for a targeted Ad vector. By demonstrating the efficient targeting of Ad with CD40L to human cancer cells and dendritic cells (DCs), Applicants highlight the advantages offered by the fiber replacement strategy for the generation of tropism-modified therapeutic vectors.

Applicants demonstrated that the incorporation of the FF/CD40L chimera into the Ad virion does not affect the functional structure of its CD40-binding component, resulting in a vector capable of infecting target cells through a CD40-mediated pathway. However, comparison of the CD40-targeted virus with untargeted Ad containing wild-type fibers showed an unfavorable 40-fold difference in transduction efficiency on 293.CD40 cells, which express CAR and CD40 at high levels. Simultaneously, the experiments with radiolabeled Ad5LucFF/CD40L and Ad5Luc1 revealed that the binding of both viruses to 293.CD40 cells was equally efficient. That result led Applicants to the hypothesis that complete deletion of the fiber in Ad5LucFF/CD40L affected its ability to accomplish a step in the infection process downstream from primary binding to the cell surface. For instance, this deletion could affect the dynamics of the escape of the virus from the endosome following internalization, as well as its intracellular trafficking. Previously published findings on the altered intracellular migration of Ad5 virions incorporating Ad serotype 7 fibers provide reasonable grounds for such an explanation (see, e.g., Miyazawa et al., 2001, J. Virol. 75:1387-1400 and Miyazawaet al., 1999, J. Virol. 73:6056-6065). To test this hypothesis, Applicants constructed a mosaic version of Ad5LucFF/CD40L which, in addition to the FF/CD40L chimera, also contained an Ad5 fiber protein unable to bind to CAR due to a mutation in the knob domain. The presence of this mutated fiber protein indeed increased the infectivity of the CD40-targeted vector to the level seen for Ad5Luc 1.

Subsequent use of Ad5LucFF/CD40L bearing either FF/CD40L alone or in combination with the mutated Ad5 fiber protein showed the superior efficacy of this vector on human monocyte-derived DCs, suggesting that it may serve as a prototype for the derivation of therapeutic vectors for genetic immunization. For instance, such vectors could be used ex vivo or in vivo for directed delivery of antigen-encoding genes to human DCs to induce the development of an antigen-specific immune response. Similarly, the fact that Ad5LucFF/CD40L proved to be far more efficacious than Ad5Luc 1 in transducing human bladder tumor cells suggests that its conditionally replicative derivatives would be rational choices as gene therapeutic agents for fighting this type of cancer.

### Example 5

### Single Chain Antibody (scFv) Ligand Incorporation into Ad

Adenoviral vectors (Ad) are of high utility for gene therapy applications owing to their capacity to accomplish highly efficient gene transfer in vitro and in vivo. In consideration of the latter capacity, Ad have been employed for a variety of human clinical gene therapy applications which embody in vivo gene delivery schemas. Indeed, adenovirus-based gene therapy interventions for cancer have achieved valid therapeutic results in human clinical trials for cancer. On this basis, adenovirus-based therapeutic agents for cancer have been clinically approved for human use as a legitimate component of the pharmacological armamentarium in Asia and are being advanced in Phase II/III trials in the USA.

Despite their emerging utility, Ad have been limited to the contexts of local and loco-regional neoplastic disease. This is due to the fact that the parent adenovirus has a promiscuous trophism resulting in the potential to transduce non-target cells, as well as target cells, relevant to disease pathobiology. Non-target cell transduction would serve to limit effective Ad dose, potentially undermining agent potency, and to induce clinical toxicity at non-target sites, potentially undermining the therapeutic index of the adenovirus agents. It is thus clear that the capacity to direct adenovirus infection exclusively to target cells would improve the therapeutic profile of adenovirus-based therapeutic interventions.

On the basis of these considerations, strategies to achieve targeted gene delivery via Ad have been endeavored via modification of viral trophism. Strategies to achieve this end have employed re-targeting “adapters” which cross-link Ad to non-native receptors characteristic of target cells. These studies have established that Ad can be routed to non-native cellular pathways, with retention of efficient gene delivery dynamics, and with the achievement of target cell specific gene delivery. Of note, the principle of targeted gene delivery via trophism modified Ad has been demonstrated in the context of in vitro models, in vivo animal models, and stringent substrate system of primary human tissue. Further, approval of targeting strategies by US Federal regulatory bodies has established the basis of incorporation of these approaches into human clinical context.

Another approach to achieve trophism modification is based on genetic capsid modification of the virion. In this regard, as Ad capsid proteins dictate the key steps of target cell binding and entry, it is logical to alter these steps by alteration of these capsid proteins. Maneuvers to alter Ad trophism via genetic capsid modification offer clear conceptual advantages from a commercial standpoint and from the perspective of regulatory approval. On this basis, efforts to accomplish Ad retargeting have been developed involving modification of adenovirus capsid proteins fiber, hexon, penton and pIX.

Strategies to achieve trophism alteration of Ad via genetic capsid modification have been based upon the concept of incorporating targeting ligands within adenovirus capsid proteins. Candidate targeting ligands include natural physiologic ligands or peptide and single chain antibody (scFv) ligands derived by genetic methods and/or bacteriophage biopanning methods. Irrespective of the source, the employment of such targeting ligands must recognize key functional requirements. Specifically, ligand incorporation into an adenovirus capsid protein must not perturb the normal quaternary structure of the capsid component or else normal viron assembly would be compromised. Further, ligands must maintain their affinity and specificity with fidelity when incorporated at the new adenovirus capsid locale.

It is noteworthy that whereas a number of capsid sites can be modified to incorporate ligands, a number of restrictions have impaired the achievement of valid cell-specific targeting via genetic capsid modification approaches. In the first regard, identified capsid sites have been relatively restrictive with respect to the size of ligand which can successfully incorporated. This is based upon structural constraints capsid proteins superimpose on ligand incorporation sites. This consideration has greatly limited the number of available targeting ligands which can be exploited for targeting purposes. Further, phage biopanning delivered peptide ligands may loose specificity/affinity in the new context of the adenovirus capsid. This loss of fidelity has limited the utility of the published repertoire of peptide targeting ligands to a very small minority thereof.

The foregoing considerations have rationalized the development of alternative approaches for genetic capsid modification. Ideally, such approaches could allow the incorporation of larger ligands which embody high affinity and specificity. Of the available candidate ligands, single chain antibodies (scFv) fulfill many of these key requirements. Of note, there are many available scFv with useful target cell specifications. Further, widely available techniques, such as phage biopanning, potentially allow derivation of new scFv with useful target cell specificities. On the basis of these principles it is apparent that an approach to accomplish genetic capsid modification of adenovirus whereby scFv could be incorporated would advance the utilities of Ad by virtue of the achievement of vector-based target cell specificity.

To address this issue Applicants have developed a genetic capsid modification approach to allow Ad incorporation of scFv. Applicants have employed a strategy of “fiber replacement” whereby the major capsid protein fiber is replaced by a chimeric molecule containing the native fiber amino terminus, to allow capsid incorporation, fused the T4 pol protein fibritin as a trimeric substitute for the fiber shaft/knob. Functional removal of the knob in this instance allows for the possibility of incorporating larger targeting ligands at the fibritin carboyx terminus without the structural constraints imposed by the fiber knob. Further, the removal of fiber knob eliminates the native trophism aspect of knob embodied within its CAR recognition domains. The fiber replacement strategy thus represents a major technical advance for the achievement of Ad retargeting via genetic capsid modification. Indeed, studies with both model “artificial receptor” systems and large native physiologic ligands have clearly established the principle that precise, cell specific targeting can be achieved via Ad subject to this trophism modification approach. Indeed, such targeted gene delivery has been demonstrated in stringent human substrate systems which have rationalized the advancement of such vectors into human clinical trials.

Recognition of the unique capacities for ligand incorporation embodied in the fiber replacement approach, Applicants speculated that this method would provide a means for scFv incorporation into Ad. Indeed, the enhanced capacity to incorporate ligands of this size was predicative of success in this endeavor. Such an achievement would link Ad targeting initiatives to the widely available targeting capacities embodied in the available repertoire of available/derivable scFv. Initial attempts to achieve scFv incorporation via the fiber replacement approach demonstrated that viable adenovirus particles could be derived which contained capsid incorporated scFv. Unfortunately, targeted gene delivery via these scFv-incorporating Ad did not demonstrate the desired specificity embodied within the unincorporated scFv. It thus appeared that scFv functionality in the context of adenovirus incorporation was not necessarily retained.

Based on the foregoing, a consideration of the biologic principles related to adenovirus incorporation of scFv was endeavored. In this regard, adenovirus capsid proteins are synthesized in the cytosol of the producer cell with nuclear assembly and maturation of capsids. Of note in this schema, there is no routing of adenovirus capsid proteins via the secretory pathway of the host cell. This is an important biologic distinction between adenovirus and the RNA virus-based gene transfer vectors, such as retrovirus and lentivirus. In these latter instances virion proteins exploit the host protein synthesis/transport mechanisms to derive key virion component proteins. The synthetic pathway of Ad, on the other hand, requires that viral protein, and any heterologous proteins incorporated for targeting purposes, retain structural and functional intergrity in the context of the distinct redox environment of the host cell cytosol and nucleus.

In this latter regard, it is noteworthy that scFv have been designed to embody many of the key attributes of their parental antibodies. In addition to their retention of the antigen recognition profile of the parent antibody, the structural arrangement of the heavy chain and light chain domains require assembly in a cellular milieu comparable to their native parental antibodies. Thus, cellular routing via the secondary export pathway of the RER is required for proper assembly/folding of scFv. This routing requirement is opposed to the routing requirements of adenovirus capsid proteins. The capsid incorporation of a targeting ligand imposes the cellular routing of the adenovirus capsid component on the incorporated ligand. In this schema, capsid incorporated scFv would undergo obligate cellular routing via the cytosol and nucleus. Of note, the redox potential of these cellular milieus is distinct from the RER normally employed for scFv synthesis and thus potentially deleterious to the proper folding and assembly required for retention of target antigen recognition.

To address this issue, Applicants considered the use of scFv which embodied resistance to the deleterious effects of routing via the adenovirus' synthetic pathways. The source of such “stabilized” scFv was embodied in diverse and non-obvious molecular engineering enterprises. In this regard, targeted functional knockouts of cellular/virus proteins via “intrabodies” has been developed as a therapeutic tool and as a means to study functional relationships within the context of cellular physiology. Such intrabodies have been developed against cellular targets in a variety of subcellular locales, including the nucleus and cytosol. Thus, defined intrabodies which successfully accomplished targeted functional knockout at these subcellular locales logically retained antigen recognition fidelity in these contexts. Such intrabodies potentially represented scFv which embody stabilization commensurate with the dictates of adenovirus capsid incorporation. In addition, efforts to directly stabilize scFv structure have been endeavored via genetic engineering methods. In these strategies a stabilized scFv “framework” is developed via directed mutations in the scFv CDR regions. These stabilized CDRs framework can then serve as a scaffold onto which scFv variable domains, which embody antigen recognition, can then be grafted by molecular engineering methods. The chimeric scFv thus manifests the desired antigen recognition profile while also embodying the stability of the scaffold CDR domain. Other methods for scFv stabilization have also been described. We hypothesized that scFv which embodied “stabilization” via any of these approaches would also manifest stability during the course of adenovirus capsid assembly that would allow retention of their key property of antigen recognition.

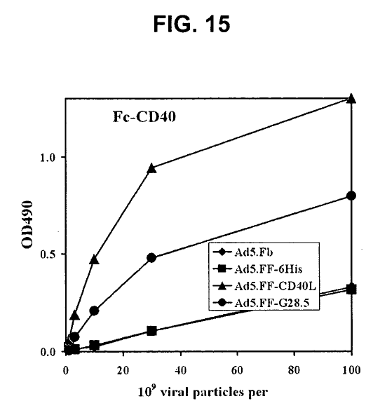

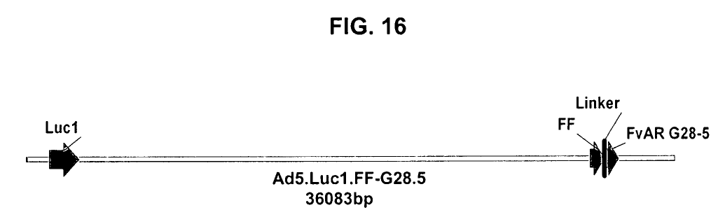

To establish the generalizability of this principle, Applicants sought to develop an adenoviral vector targetable via a stabilized scFv incorporated into the capsid via the fiber replacement approach. Applicants initially developed an scFv targeted to CD40, a cell surface marker characteristic of normal immunoregulatory cells and also a marker of neoplastic lymphoreticular and epithelial neoplasms. An anti-CD40 scFv was derived by phage biopanning methods. The anti-CD40 scFv was then engineered to achieve molecular stabilization via modification of the CDR scaffold, as noted above. A cDNA encoding the stabilized scFv was then incorporated into a chimeric fiber construct for employment via fiber replacement genetic capsid engineering. As shown in FIG. 15, rescued adenoviral particles demonstrated successful incorporation of the scFv as demonstrated by ELISA assay whereby scFv recognition of the target antigen was apparent.

### Example 6

### Sequence of Ad5.Luc1.FF-28.51 (SEQ ID NO: 21)

| | |
|---|---|
| 1 | catcatcaat aatatacctt attttggatt gaagccaata tgataatgag ggggtggagt |
| 61 | ttgtgacgtg gcgcggggcg tgggaacggg gcgggtgacg tagtagtgtg gcggaagtgt |
| 121 | gatgttgcaa gtgtggcgga acacatgtaa gcgacggatg tggcaaaagt gacgtttttg |
| 181 | gtgtgcgccg gtgtacacag gaagtgacaa ttttcgcgcg gttttaggcg gatgttgtag |
| 241 | taaatttggg cgtaaccgag taagatttgg ccattttcgc gggaaaactg aataagagga |
| 301 | agtgaaatct gaataatttt gtgttactca tagcgcgtaa tatttgtcta gggccgcggg |
| 361 | gactttgacc gtttacgtgg agactcgccc aggtgttttt ctcaggtgtt ttccgcgttc |
| 421 | cgggtcaaag ttggcgtttt attattatag tcactctagg cggccgcgat ctatacattg |
| 481 | aatcaatatt ggcaattagc catattagtc attggttata tagcataaat caatattggc |
| 541 | tattggccat tgcatacgtt gtatctatat cataatatgt acatttatat tggctcatgt |
| 601 | ccaatatgac cgccatgttg acattgatta ttgactagtt attaatagta atcaattacg |
| 661 | gggtcattag ttcatagccc atatatggag ttccgcgtta cataacttac ggtaaatggc |
| 721 | ccgcctggct gaccgcccaa cgacccccgc ccattgacgt caataatgag ctatgttccc |
| 781 | atagtaacgc caatagggac tttccattga cgtcaatggg tggagtattt acggtaaact |
| 841 | gcccacttgg cagtacatca agtgtatcat atgccaagtc cgccccctat tgacgtcaat |
| 901 | gacggtaaat ggcccgcctg gcattatgcc cagtacatga ccttacggga ctttcctact |
| 961 | tggcagtaca tctacgtatt agtcatcgct attaccatgg tgatgcggtt ttggcagtac |
| 1021 | accaatgggc gtggatagcg gtttgactca cggggatttc caagtctcca ccccattgac |
| 1081 | gtcaatggga gtttgttttg gcaccaaaat caacgggact ttccaaaatg tcgtaataac |
| 1141 | cccgccccgt tgacgcaaat gggcggtagg cgtgtacggt gggaggtcta tataagcaga |
| 1201 | gctcgtttag tgaaccgtca gatccggtcg cgcgaattga tccaaatgga agacgccaaa |
| 1261 | aacataaaga aaggcccggc gccattctat cctctagagg atggaaccgc tggagagcaa |
| 1321 | ctgcataagg ctatgaagag atacgccctg gttcctggaa caattgcttt tacagatgca |
| 1381 | catatcgagg tgaacatcac gtacgcggaa tacttcgaaa tgtccgttcg gttggcagaa |
| 1441 | gctatgaaac gatatgggct gaatacaaat cacagaatcg tcgtatgcag tgaaaactct |
| 1501 | cttcaattct ttatgccggt gttgggcgcg ttatttatcg gagttgcagt tgcgcccgcg |
| 1561 | aacgacattt ataatgaacg tgaattgctc aacagtatga acatttcgca gcctaccgta |
| 1621 | gtgtttgttt ccaaaaaggg gttgcaaaaa attttgaacg tgcaaaaaaa attaccaata |
| 1681 | atccagaaaa ttattatcat ggattctaaa acggattacc agggatttca gtcgatgtac |
| 1741 | acgttcgtca catctcatct acctcccggt tttaatgaat acgattttgt accagagtcc |
| 1801 | tttgatcgtg acaaaacaat tgcactgata atgaattcct ctggatctac tgggttacct |
| 1861 | aagggtgtgg cccttccgca tagaactgcc tgcgtcagat tctcgcatgc cagagatcct |
| 1921 | atttttggca atcaaatcat tccggatact gcgattttaa gtgttgttcc attccatcac |
| 1981 | ggttttggaa tgtttactac actcggatat ttgatatgtg gatttcgagt cgtcttaatg |
| 2041 | tatagatttg aagaagagct gtttttacga tcccttcagg attacaaaat tcaaagtgcg |
| 2101 | ttgctagtac caaccctatt ttcattcttc gccaaaagca ctctgattga caaatacgat |
| 2161 | ttatctaatt tacacgaaat tgcttctggg ggcgcacctc tttcgaaaga agtcggggaa |
| 2221 | gcggttgcaa aacgcttcca tcttccaggg atacgacaag gatatgggct cactgagact |
| 2281 | acatcagcta ttctgattac acccgagggg gatgataaac cgggcgcggt cggtaaagtt |
| 2341 | gttccatttt ttgaagcgaa ggttgtggat ctggataccg ggaaaacgct gggcgttaat |
| 2401 | cagagaggcg aattatgtgt cagaggacct atgattatgt ccggttatgt aaacaatccg |
| 2461 | gaagcgacca acgccttgat tgacaaggat ggatggctac attctggaga catagcttac |
| 2521 | tgggacgaag acgaacactt cttcatagtt gaccgcttga agtctttaat caaatacaaa |
| 2581 | ggatatcagg tggcccccgc tgaattggag tcgatattgt tacaacaccc caacatcttc |
| 2641 | gacgcgggcg tggcaggtct tcccgacgat gacgccggtg aacttcccgc cgccgttgtt |
| 2701 | gttttggagc acggaaagac gatgacggaa aaagagatcg tggattacgt cgccagtcaa |
| 2761 | gtaacaaccg cgaaaaagtt gcgcggagga gttgtgtttg tggacgaagt accgaaaggt |
| 2821 | cttaccggaa aactcgacgc aagaaaaatc agagagatcc tcataaaggc caagaagggc |
| 2881 | ggaaagtcca aattgtaaaa tgtaactgta ttcagcgatg acgaaattct tagctattgt |
| 2941 | aatcctccga ggcctcgacc tgcaggcatg caagcttggg atctttgtga aggaacctta |
| 3001 | cttctgtggt gtgacataat tggacaaact acctacagag atttaaagct ctaaggtaaa |
| 3061 | tataaaattt ttaagtgtat aatgtgttaa actactgatt ctaattgttt gtgtatttta |
| 3121 | gattcacagt cccaaggctc atttcaggcc cctcagtcct cacagtctgt tcatgatcat |
| 3181 | aatcagccat accacatttg tagaggtttt acttgcttta aaaaacctcc cacacctccc |
| 3241 | cctgaacctg aaacataaaa tgaatgcaat tgttgttgtt aacttgttta ttgcagctta |
| 3301 | taatggttac aaataaagca atagcatcac aaatttcaca aataaagcat ttttttcact |
| 3361 | gcattctagt tgtggtttgt ccaaactcat caatgtatct tatcatgtct ggatcgcggc |
| 3421 | cgcctagagg gaaggtgctg aggtacgatg agacccgcac caggtgcaga ccctgcgagt |
| 3481 | gtggcggtaa acatattagg aaccagcctg tgatgctgga tgtgaccgag gagctgaggc |
| 3541 | ccgatcactt ggtgctggcc tgcacccgcg ctgagtttgg ctctagcgat gaagatacag |
| 3601 | attgaggtac tgaaatgtgt gggcgtggct taagggtggg aaagaatata taaggtgggg |
| 3661 | gtcttatgta gttttgtatc tgttttgcag cagccgccgc cgccatgagc accaactcgt |
| 3721 | ttgatggaag cattgtgagc tcatatttga caacgcgcat gcccccatgg gccggggtgc |
| 3781 | gtcagaatgt gatgggctcc agcattgatg gtcgccccgt cctgcccgca aactctacta |
| 3841 | ccttgaccta cgagaccgtg tctggaacgc cgttggagac tgcagcctcc gccgccgctt |
| 3901 | cagccgctgc agccaccgcc cgcgggattg tgactgactt tgctttcctg agcccgcttg |
| 3961 | caagcagtgc agcttcccgt tcatccgccc gcgatgacaa gttgacggct cttttggcac |
| 4021 | aattggattc tttgacccgg gaacttaatg tcgtttctca gcagctgttg gatctgcgcc |
| 4081 | agcaggtttc tgccctgaag gcttcctccc ctcccaatgc ggtttaaaac ataaataaaa |
| 4141 | aaccagactc tgtttggatt tggatcaagc aagtgtcttg ctgtctttat ttaggggttt |
| 4201 | tgcgcgcgcg gtaggcccgg gaccagcggt ctcggtcgtt gagggtcctg tgtatttttt |
| 4261 | ccaggacgtg gtaaaggtga ctctggatgt tcagatacat gggcataagc ccgtctctgg |
| 4321 | ggtggaggta gcaccactgc agagcttcat gctgcggggt ggtgttgtag atgatccagt |
| 4381 | cgtagcagga gcgctgggcg tggtgcctaa aaatgtcttt cagtagcaag ctgattgcca |
| 4441 | ggggcaggcc cttggtgtaa gtgtttacaa agcggttaag ctgggatggg tgcatacgtg |
| 4501 | gggatatgag atgcatcttg gactgtattt ttaggttggc tatgttccca gccatatccc |
| 4561 | tccggggatt catgttgtgc agaaccacca gcacagtgta tccggtgcac ttgggaaatt |
| 4621 | tgtcatgtag cttagaagga aatgcgtgga agaacttgga gacgcccttg tgacctccaa |
| 4681 | gattttccat gcattcgtcc ataatgatgg caatgggccc acgggcggcg gcctgggcga |
| 4741 | agatatttct gggatcacta acgtcatagt tgtgttccag gatgagatcg tcataggcca |
| 4801 | tttttacaaa gcgcgggcgg agggtgccag actgcggtat aatggttcca tccggcccag |
| 4861 | gggcgtagtt accctcacag atttgcattt cccacgcttt gagttcagat ggggggatca |
| 4921 | tgtctacctg cggggcgatg aagaaaacgg tttccggggt aggggagatc agctgggaag |
| 4981 | aaagcaggtt cctgagcagc tgcgacttac cgcagccggt gggcccgtaa atcacaccta |
| 5041 | ttaccgggtg caactggtag ttaagagagc tgcagctgcc gtcatccctg agcagggggg |
| 5101 | ccacttcgtt aagcatgtcc ctgactcgca tgttttccct gaccaaatcc gccagaaggc |
| 5161 | gctcgccgcc cagcgatagc agttcttgca aggaagcaaa gtttttcaac ggtttgagac |
| 5221 | cgtccgccgt aggcatgctt ttgagcgttt gaccaagcag ttccaggcgg tcccacagct |
| 5281 | cggtcacctg ctctacggca tctcgatcca gcatatctcc tcgtttcgcg ggttggggcg |
| 5341 | gctttcgctg tacggcagta gtcggtgctc gtccagacgg gccagggtca tgtctttcca |
| 5401 | cgggcgcagg gtcctcgtca gcgtagtctg ggtcacggtg aaggggtgcg ctccgggctg |
| 5461 | cgcgctggcc agggtgcgct tgaggctggt cctgctggtg ctgaagcgct gccggtcttc |
| 5521 | gccctgcgcg tcggccaggt agcatttgac catggtgtca tagtccagcc cctccgcggc |
| 5581 | gtggcccttg gcgcgcagct tgcccttgga ggaggcgccg cacgaggggc agtgcagact |
| 5641 | tttgagggcg tagagcttgg gcgcgagaaa taccgattcc ggggagtagg catccgcgcc |
| 5701 | gcaggccccg cagacggtct cgcattccac gagccaggtg agctctggcc gttcggggtc |
| 5761 | aaaaaccagg tttcccccat gctttttgat gcgtttctta cctctggttt ccatgagccg |
| 5821 | gtgtccacgc tcggtgacga aaaggctgtc cgtgtccccg tatacagact tgagaggcct |
| 5881 | gtcctcgagc ggtgttccgc ggtcctcctc gtatagaaac tcggaccact ctgagacaaa |
| 5941 | ggctcgcgtc caggccagca cgaaggaggc taagtgggag gggtagcggt cgttgtccac |
| 6001 | tagggggtcc actcgctcca gggtgtgaag acacatgtcg ccctcttcgg catcaaggaa |
| 6061 | ggtgattggt ttgtaggtgt aggccacgtg accgggtgtt cctgaagggg ggctataaaa |
| 6121 | gggggtgggg gcgcgttcgt cctcactctc ttccgcatcg ctgtctgcga gggccagctg |
| 6181 | ttggggtgag tactccctct gaaaagcggg catgacttct gcgctaagat tgtcagtttc |
| 6241 | caaaaacgag gaggatttga tattcacctg gcccgcggtg atgcctttga gggtggccgc |
| 6301 | atccatctgg tcagaaaaga caatcttttt gttgtcaagc ttggtggcaa acgacccgta |
| 6361 | gagggcgttg gacagcaact tggcgatgga gcgcagggtt tggtttttgt cgcgatcggc |
| 6421 | gcgctccttg gccgcgatgt ttagctgcac gtattcgcgc gcaacgcacc gccattcggg |
| 6481 | aaagacggtg gtgcgctcgt cgggcaccag gtgcacgcgc caaccgcggt tgtgcagggt |
| 6541 | gacaaggtca acgctggtgg ctacctctcc gcgtaggcgc tcgttggtcc agcagaggcg |
| 6601 | gccgcccttg cgcgagcaga atggcggtag ggggtctagc tgcgtctcgt ccggggggtc |
| 6661 | tgcgtccacg gtaaagaccc cgggcagcag gcgcgcgtcg aagtagtcta tcttgcatcc |
| 6721 | ttgcaagtct agcgcctgct gccatgcgcg ggcggcaagc gcgcgctcgt atgggttgag |
| 6781 | tgggggaccc catggcatgg ggtgggtgag cgcggaggcg tacatgccgc aaatgtcgta |
| 6841 | aacgtagagg ggctctctga gtattccaag atatgtaggg tagcatcttc caccgcggat |
| 6901 | gctggcgcgc acgtaatcgt atagttcgtg cgagggagcg aggaggtcgg gaccgaggtt |
| 6961 | gctacgggcg ggctgctctg ctcggaagac tatctgcctg aagatggcat gtgagttgga |
| 7021 | tgatatggtt ggacgctgga agacgttgaa gctggcgtct gtgagaccta ccgcgtcacg |
| 7081 | cacgaaggag gcgtaggagt cgcgcagctt gttgaccagc tcggcggtga cctgcacgtc |
| 7141 | tagggcgcag tagtccaggg tttccttgat gatgtcatac ttatcctgtc cctttttttt |
| 7201 | ccacagctcg cggttgagga caaactcttc gcggtctttc cagtactctt ggatcggaaa |
| 7261 | cccgtcggcc tccgaacggt aagagcctag catgtagaac tggttgacgg cctggtaggc |
| 7321 | gcagcatccc ttttctacgg gtagcgcgta tgcctgcgcg gccttccgga gcgaggtgtg |
| 7381 | ggtgagcgca aaggtgtccc tgaccatgac tttgaggtac tggtatttga agtcagtgtc |
| 7441 | gtcgcatccg ccctgctccc agagcaaaaa gtccgtgcgc tttttggaac gcggatttgg |
| 7501 | cagggcgaag gtgacatcgt tgaagagtat ctttcccgcg cgaggcataa agttgcgtgt |
| 7561 | gatgcggaag ggtcccggca cctcggaacg gttgttaatt acctgggcgg cgagcacgat |
| 7621 | ctcgtcaaag ccgttgatgt tgtggcccac aatgtaaagt tccaagaagc gcgggatgcc |
| 7681 | cttgatggaa ggcaattttt taagttcctc gtaggtgagc tcttcagggg agctgagccc |
| 7741 | gtgctctgaa agggcccagt ctgcaagatg agggttggaa gcgacgaatg agctccacag |
| 7801 | gtcacgggcc attagcattt gcaggtggtc gcgaaaggtc ctaaactggc gacctatggc |
| 7861 | cattttttct ggggtgatgc agtagaaggt aagcgggtct tgttcccagc ggtcccatcc |
| 7921 | aaggttcgcg gctaggtctc gcgcggcagt cactagaggc tcatctccgc cgaacttcat |
| 7981 | gaccagcatg aagggcacga gctgcttccc aaaggccccc atccaagtat aggtctctac |
| 8041 | atcgtaggtg acaaagagac gctcggtgcg aggatgcgag ccgatcggga agaactggat |
| 8101 | ctcccgccac caattggagg agtggctatt gatgtggtga aagtagaagt ccctgcgacg |
| 8161 | ggccgaacac tcgtgctggc ttttgtaaaa acgtgcgcag tactggcagc ggtgcacggg |
| 8221 | ctgtacatcc tgcacgaggt tgacctgacg accgcgcaca aggaagcaga gtgggaattt |
| 8281 | gagcccctcg cctggcgggt ttggctggtg gtcttctact tcggctgctt gtccttgacc |
| 8341 | gtctggctgc tcgaggggag ttacggtgga tcggaccacc acgccgcgcg agcccaaagt |
| 8401 | ccagatgtcc gcgcgcggcg gtcggagctt gatgacaaca tcgcgcagat gggagctgtc |
| 8461 | catggtctgg agctcccgcg gcgtcaggtc aggcgggagc tcctgcaggt ttacctcgca |
| 8521 | tagacgggtc agggcgcggg ctagatccag gtgataccta atttccaggg gctggttggt |
| 8581 | ggcggcgtcg atggcttgca agaggccgca tccccgcggc gcgactacgg taccgcgcgg |
| 8641 | cgggcggtgg gccgcggggg tgtccttgga tgatgcatct aaaagcggtg acgcgggcga |
| 8701 | gcccccggag gtaggggggg ctccggaccc gccgggagag ggggcagggg cacgtcggcg |
| 8761 | ccgcgcgcgg gcaggagctg gtgctgcgcg cgtaggttgc tggcgaacgc gacgacgcgg |
| 8821 | cggttgatct cctgaatctg gcgcctctgc gtgaagacga cgggcccggt gagcttgagc |
| 8881 | ctgaaagaga gttcgacaga atcaatttcg gtgtcgttga cggcggcctg gcgcaaaatc |
| 8941 | tcctgcacgt ctcctgagtt gtcttgatag gcgatctcgg ccatgaactg ctcgatctct |
| 9001 | tcctcctgga gatctccgcg tccggctcgc tccacggtgg cggcgaggtc gttggaaatg |
| 9061 | cgggccatga gctgcgagaa ggcgttgagg cctccctcgt tccagacgcg gctgtagacc |
| 9121 | acgccccctt cggcatcgcg ggcgcgcatg accacctgcg cgagattgag ctccacgtgc |
| 9181 | cgggcgaaga cggcgtagtt tcgcaggcgc tgaaagaggt agttgagggt ggtggcggtg |
| 9241 | tgttctgcca cgaagaagta cataacccag cgtcgcaacg tggattcgtt gatatccccc |
| 9301 | aaggcctcaa ggcgctccat ggcctcgtag aagtccacgg cgaagttgaa aaactgggag |
| 9361 | ttgcgcgccg acacggttaa ctcctcctcc agaagacgga tgagctcggc gacagtgtcg |
| 9421 | cgcacctcgc gctcaaaggc tacaggggcc tcttcttctt cttcaatctc ctcttccata |
| 9481 | agggcctccc cttcttcttc ttctggcggc ggtgggggag gggggacacg gcggcgacga |
| 9541 | cggcgcaccg ggaggcggtc gacaaagcgc tcgatcatct ccccgcggcg acggcgcatg |
| 9601 | gtctcggtga cggcgcggcc gttctcgcgg gggcgcagtt ggaagacgcc gcccgtcatg |
| 9661 | tcccggttat gggttggcgg ggggctgcca tgcggcaggg atacggcgct aacgatgcat |
| 9721 | ctcaacaatt gttgtgtagg tactccgccg ccgagggacc tgagcgagtc cgcatcgacc |
| 9781 | ggatcggaaa acctctcgag aaaggcgtct aaccagtcac agtcgcaagg taggctgagc |
| 9841 | accgtggcgg gcggcagcgg gcggcggtcg gggttgtttc tggcggaggt gctgctgatg |
| 9901 | atgtaattaa agtaggcggt cttgagacgg cggatggtcg acagaagcac catgtccttg |
| 9961 | ggtccggcct gctgaatgcg caggcggtcg gccatgcccc aggcttcgtt ttgacatcgg |
| 10021 | cgcaggtctt tgtagtagtc ttgcatgagc ctttctaccg gcacttcttc ttctccttcc |
| 10081 | tcttgtcctg catctcttgc atctatcgct gcggcggcgg cggagtttgg ccgtaggtgg |
| 10141 | cgccctcttc ctcccatgcg tgtgaccccg aagcccctca tcggctgaag cagggctagg |
| 10201 | tcggcgacaa cgcgctcggc taatatggcc tgctgcacct gcgtgagggt agactggaag |
| 10261 | tcatccatgt ccacaaagcg gtggtatgcg cccgtgttga tggtgtaagt gcagttggcc |
| 10321 | ataacggacc agttaacggt ctggtgaccc ggctgcgaga gctcggtgta cctgagacgc |
| 10381 | gagtaagccc tcgagtcaaa tacgtagtcg ttgcaagtcc gcaccaggta ctggtatccc |
| 10441 | accaaaaagt gcggcggcgg ctggcggtag aggggccagc gtagggtggc cggggctccg |
| 10501 | ggggcgagat cttccaacat aaggcgatga tatccgtaga tgtacctgga catccaggtg |
| 10561 | atgccggcgg cggtggtgga ggcgcgcgga aagtcgcgga cgcggttcca gatgttgcgc |
| 10621 | agcggcaaaa agtgctccat ggtcgggacg ctctggccgg tcaggcgcgc gcaatcgttg |
| 10681 | acgctctaga ccgtgcaaaa ggagagcctg taagcgggca ctcttccgtg gtctggtgga |
| 10741 | taaattcgca agggtatcat ggcggacgac cggggttcga gccccgtatc cggccgtccg |
| 10801 | ccgtgatcca tgcggttacc gcccgcgtgt cgaacccagg tgtgcgacgt cagacaacgg |
| 10861 | gggagtgctc cttttggctt ccttccaggc gcggcggctg ctgcgctagc ttttttggcc |
| 10921 | actggccgcg cgcagcgtaa gcggttaggc tggaaagcga aagcattaag tggctcgctc |
| 10981 | cctgtagccg gagggttatt ttccaagggt tgagtcgcgg gacccccggt tcgagtctcg |
| 11041 | gaccggccgg actgcggcga acgggggttt gcctccccgt catgcaagac cccgcttgca |
| 11101 | aattcctccg gaaacaggga cgagcccctt ttttgctttt cccagatgca tccggtgctg |
| 11161 | cggcagatgc gcccccctcc tcagcagcgg caagagcaag agcagcggca gacatgcagg |
| 11221 | gcaccctccc ctcctcctac cgcgtcagga ggggcgacat ccgcggttga cgcggcagca |
| 11281 | gatggtgatt acgaaccccc gcggcgccgg gcccggcact acctggactt ggaggagggc |
| 11341 | gagggcctgg cgcggctagg agcgccctct cctgagcggt acccaagggt gcagctgaag |
| 11401 | cgtgatacgc gtgaggcgta cgtgccgcgg cagaacctgt ttcgcgaccg cgagggagag |
| 11461 | gagcccgagg agatgcggga tcgaaagttc cacgcagggc gcgagctgcg gcatggcctg |
| 11521 | aatcgcgagc ggttgctgcg cgaggaggac tttgagcccg acgcgcgaac cgggattagt |
| 11581 | cccgcgcgcg cacacgtggc ggccgccgac ctggtaaccg catacgagca gacggtgaac |
| 11641 | caggagatta actttcaaaa aagctttaac aaccacgtgc gtacgcttgt ggcgcgcgag |
| 11701 | gaggtggcta taggactgat gcatctgtgg gactttgtaa gcgcgctgga gcaaaaccca |
| 11761 | aatagcaagc cgctcatggc gcagctgttc cttatagtgc agcacagcag ggacaacgag |
| 11821 | gcattcaggg atgcgctgct aaacatagta gagcccgagg gccgctggct gctcgatttg |
| 11881 | ataaacatcc tgcagagcat agtggtgcag gagcgcagct tgagcctggc tgacaaggtg |
| 11941 | gccgccatca actattccat gcttagcctg ggcaagtttt acgcccgcaa gatataccat |
| 12001 | accccttacg ttcccataga caaggaggta aagatcgagg ggttctacat gcgcatggcg |
| 12061 | ctgaaggtgc ttaccttgag cgacgacctg ggcgtttatc gcaacgagcg catccacaag |
| 12121 | gccgtgagcg tgagccggcg gcgcgagctc agcgaccgcg agctgatgca cagcctgcaa |
| 12181 | agggccctgg ctggcacggg cagcggcgat agagaggccg agtcctactt tgacgcgggc |
| 12241 | gctgacctgc gctgggcccc aagccgacgc gccctggagg cagctggggc cggacctggg |
| 12301 | ctggcggtgg cacccgcgcg cgctggcaac gtcggcggcg tggaggaata tgacgaggac |
| 12361 | gatgagtacg agccagagga cggcgagtac taagcggtga tgtttctgat cagatgatgc |
| 12421 | aagacgcaac ggacccggcg gtgcgggcgg cgctgcagag ccagccgtcc ggccttaact |
| 12481 | ccacggacga ctggcgccag gtcatggacc gcatcatgtc gctgactgcg cgcaatcctg |
| 12541 | acgcgttccg gcagcagccg caggccaacc ggctctccgc aattctggaa gcggtggtcc |
| 12601 | cggcgcgcgc aaaccccacg cacgagaagg tgctggcgat cgtaaacgcg ctggccgaaa |
| 12661 | acagggccat ccggcccgac gaggccggcc tggtctacga cgcgctgctt cagcgcgtgg |
| 12721 | ctcgttacaa cagcggcaac gtgcagacca acctggaccg gctggtgggg gatgtgcgcg |
| 12781 | aggccgtggc gcagcgtgag cgcgcgcagc agcagggcaa cctgggctcc atggttgcac |
| 12841 | taaacgcctt cctgagtaca cagcccgcca acgtgccgcg gggacaggag gactacacca |
| 12901 | actttgtgag cgcactgcgg ctaatggtga ctgagacacc gcaaagtgag gtgtaccagt |
| 12961 | ctgggccaga ctattttttc cagaccagta gacaaggcct gcagaccgta aacctgagcc |
| 13021 | aggctttcaa aaacttgcag gggctgtggg gggtgcgggc tcccacaggc gaccgcgcga |
| 13081 | ccgtgtctag cttgctgacg cccaactcgc gcctgttgct gctgctaata gcgcccttca |
| 13141 | cggacagtgg cagcgtgtcc cgggacacat acctaggtca cttgctgaca ctgtaccgcg |
| 13201 | aggccatagg tcaggcgcat gtggacgagc atactttcca ggagattaca agtgtcagcc |
| 13261 | gcgcgctggg gcaggaggac acgggcagcc tggaggcaac cctaaactac ctgctgacca |
| 13321 | accggcggca gaagatcccc tcgttgcaca gtttaaacag cgaggaggag cgcattttgc |
| 13381 | gctacgtgca gcagagcgtg agccttaacc tgatgcgcga cggggtaacg cccagcgtgg |
| 13441 | cgctggacat gaccgcgcgc aacatggaac cgggcatgta tgcctcaaac cggccgttta |
| 13501 | tcaaccgcct aatggactac ttgcatcgcg cggccgccgt gaaccccgag tatttcacca |
| 13561 | atgccatctt gaacccgcac tggctaccgc cccctggttt ctacaccggg ggattcgagg |
| 13621 | tgcccgaggg taacgatgga ttcctctggg acgacataga cgacagcgtg ttttccccgc |
| 13681 | aaccgcagac cctgctagag ttgcaacagc gcgagcaggc agaggcggcg ctgcgaaagg |
| 13741 | aaagcttccg caggccaagc agcttgtccg atctaggcgc tgcggccccg cggtcagatg |
| 13801 | ctagtagccc atttccaagc ttgatagggt ctcttaccag cactcgcacc acccgcccgc |
| 13861 | gcctgctggg cgaggaggag tacctaaaca actcgctgct gcagccgcag cgcgaaaaaa |
| 13921 | acctgcctcc ggcatttccc aacaacggga tagagagcct agtggacaag atgagtagat |
| 13981 | ggaagacgta cgcgcaggag cacagggacg tgccaggccc gcgcccgccc acccgtcgtc |
| 14041 | aaaggcacga ccgtcagcgg ggtctggtgt gggaggacga tgactcggca gacgacagca |
| 14101 | gcgtcctgga tttgggaggg agtggcaacc cgtttgcgca ccttcgcccc aggctgggga |
| 14161 | gaatgtttta aaaaaaaaaa agcatgatgc aaaataaaaa actcaccaag gccatggcac |
| 14221 | cgagcgttgg ttttcttgta ttccccttag tatgcggcgc gcggcgatgt atgaggaagg |
| 14281 | tcctcctccc tcctacgaga gtgtggtgag cgcggcgcca gtggcggcgg cgctgggttc |
| 14341 | tcccttcgat gctcccctgg acccgccgtt tgtgcctccg cggtacctgc ggcctaccgg |
| 14401 | ggggagaaac agcatccgtt actctgagtt ggcaccccta ttcgacacca cccgtgtgta |
| 14461 | cctggtggac aacaagtcaa cggatgtggc atccctgaac taccagaacg accacagcaa |
| 14521 | ctttctgacc acggtcattc aaaacaatga ctacagcccg ggggaggcaa gcacacagac |
| 14581 | catcaatctt gacgaccggt cgcactgggg cggcgacctg aaaaccatcc tgcataccaa |
| 14641 | catgccaaat gtgaacgagt tcatgtttac caataagttt aaggcgcggg tgatggtgtc |
| 14701 | gcgcttgcct actaaggaca atcaggtgga gctgaaatac gagtgggtgg agttcacgct |
| 14761 | gcccgagggc aactactccg agaccatgac catagacctt atgaacaacg cgatcgtgga |
| 14821 | gcactacttg aaagtgggca gacagaacgg ggttctggaa agcgacatcg gggtaaagtt |
| 14881 | tgacacccgc aacttcagac tggggtttga ccccgtcact ggtcttgtca tgcctggggt |
| 14941 | atatacaaac gaagccttcc atccagacat cattttgctg ccaggatgcg gggtggactt |
| 15001 | cacccacagc cgcctgagca acttgttggg catccgcaag cggcaaccct tccaggaggg |
| 15061 | ctttaggatc acctacgatg atctggaggg tggtaacatt cccgcactgt tggatgtgga |
| 15121 | cgcctaccag gcgagcttga aagatgacac cgaacagggc gggggtggcg caggcggcag |
| 15181 | caacagcagt ggcagcggcg cggaagagaa ctccaacgcg gcagccgcgg caatgcagcc |
| 15241 | ggtggaggac atgaacgatc atgccattcg cggcgacacc tttgccacac gggctgagga |
| 15301 | gaagcgcgct gaggccgaag cagcggccga agctgccgcc cccgctgcgc aacccgaggt |
| 15361 | cgagaagcct cagaagaaac cggtgatcaa acccctgaca gaggacagca agaaacgcag |
| 15421 | ttacaaccta ataagcaatg acagcacctt cacccagtac cgcagctggt accttgcata |
| 15481 | caactacggc gaccctcaga ccggaatccg ctcatggacc ctgctttgca ctcctgacgt |
| 15541 | aacctgcggc tcggagcagg tctactggtc gttgccagac atgatgcaag accccgtgac |
| 15601 | cttccgctcc acgcgccaga tcagcaactt tccggtggtg ggcgccgagc tgttgcccgt |
| 15661 | gcactccaag agcttctaca acgaccaggc cgtctactcc caactcatcc gccagtttac |
| 15721 | ctctctgacc cacgtgttca atcgctttcc cgagaaccag attttggcgc gcccgccagc |
| 15781 | ccccaccatc accaccgtca gtgaaaacgt tcctgctctc acagatcacg ggacgctacc |
| 15841 | gctgcgcaac agcatcggag gagtccagcg agtgaccatt actgacgcca gacgccgcac |
| 15901 | ctgcccctac gtttacaagg ccctgggcat agtctcgccg cgcgtcctat cgagccgcac |
| 15961 | tttttgagca agcatgtcca tccttatatc gcccagcaat aacacaggct ggggcctgcg |
| 16021 | cttcccaagc aagatgtttg gcggggccaa gaagcgctcc gaccaacacc cagtgcgcgt |
| 16081 | gcgcgggcac taccgcgcgc cctggggcgc gcacaaacgc ggccgcactg ggcgcaccac |
| 16141 | cgtcgatgac gccatcgacg cggtggtgga ggaggcgcgc aactacacgc ccacgccgcc |
| 16201 | accagtgtcc acagtggacg cggccattca gaccgtggtg cgcggagccc ggcgctatgc |
| 16261 | taaaatgaag agacggcgga ggcgcgtagc acgtcgccac cgccgccgac ccggcactgc |
| 16321 | cgcccaacgc gcggcggcgg ccctgcttaa ccgcgcacgt cgcaccggcc gacgggcggc |
| 16381 | catgcgggcc gctcgaaggc tggccgcggg tattgtcact gtgcccccca ggtccaggcg |
| 16441 | acgagcggcc gccgcagcag ccgcggccat tagtgctatg actcagggtc gcaggggcaa |
| 16501 | cgtgtattgg gtgcgcgact cggttagcgg cctgcgcgtg cccgtgcgca cccgcccccc |
| 16561 | gcgcaactag attgcaagaa aaaactactt agactcgtac tgttgtatgt atccagcggc |
| 16621 | ggcggcgcgc aacgaagcta tgtccaagcg caaaatcaaa gaagagatgc tccaggtcat |
| 16681 | cgcgccggag atctatggcc ccccgaagaa ggaagagcag gattacaagc cccgaaagct |
| 16741 | aaagcgggtc aaaaagaaaa agaaagatga tgatgatgaa cttgacgacg aggtggaact |
| 16801 | gctgcacgct accgcgccca ggcgacgggt acagtggaaa ggtcgacgcg taaaacgtgt |
| 16861 | tttgcgaccc ggcaccaccg tagtctttac gcccggtgag cgctccaccc gcacctacaa |
| 16921 | gcgcgtgtat gatgaggtgt acggcgacga ggacctgctt gagcaggcca acgagcgcct |
| 16981 | cggggagttt gcctacggaa agcggcataa ggacatgctg gcgttgccgc tggacgaggg |
| 17041 | caacccaaca cctagcctaa agcccgtaac actgcagcag gtgctgcccg cgcttgcacc |
| 17101 | gtccgaagaa aagcgcggcc taaagcgcga gtctggtgac ttggcaccca ccgtgcagct |
| 17161 | gatggtaccc aagcgccagc gactggaaga tgtcttggaa aaaatgaccg tggaacctgg |
| 17221 | gctggagccc gaggtccgcg tgcggccaat caagcaggtg gcgccgggac tgggcgtgca |
| 17281 | gaccgtggac gttcagatac ccactaccag tagcaccagt attgccaccg ccacagaggg |
| 17341 | catggagaca caaacgtccc cggttgcctc agcggtggcg gatgccgcgg tgcaggcggt |
| 17401 | cgctgcggcc gcgtccaaga cctctacgga ggtgcaaacg gacccgtgga tgtttcgcgt |
| 17461 | ttcagccccc cggcgcccgc gcggttcgag gaagtacggc gccgccagcg cgctactgcc |
| 17521 | cgaatatgcc ctacatcctt ccattgcgcc tacccccggc tatcgtggct acacctaccg |
| 17581 | ccccagaaga cgagcaacta cccgacgccg aaccaccact ggaacccgcc gccgccgtcg |
| 17641 | ccgtcgccag cccgtgctgg ccccgatttc cgtgcgcagg gtggctcgcg aaggaggcag |
| 17701 | gaccctggtg ctgccaacag cgcgctacca ccccagcatc gtttaaaagc cggtctttgt |
| 17761 | ggttcttgca gatatggccc tcacctgccg cctccgtttc ccggtgccgg gattccgagg |
| 17821 | aagaatgcac cgtaggaggg gcatggccgg ccacggcctg acgggcggca tgcgtcgtgc |
| 17881 | gcaccaccgg cggcggcgcg cgtcgcaccg tcgcatgcgc ggcggtatcc tgcccctcct |
| 17941 | tattccactg atcgccgcgg cgattggcgc cgtgcccgga attgcatccg tggccttgca |
| 18001 | ggcgcagaga cactgattaa aaacaagttg catgtggaaa aatcaaaata aaaagtctgg |
| 18061 | actctcacgc tcgcttggtc ctgtaactat tttgtagaat ggaagacatc aactttgcgt |
| 18121 | ctctggcccc gcgacacggc tcgcgcccgt tcatgggaaa ctggcaagat atcggcacca |
| 18181 | gcaatatgag cggtggcgcc ttcagctggg gctcgctgtg gagcggcatt aaaaatttcg |
| 18241 | gttccaccgt taagaactat ggcagcaagg cctggaacag cagcacaggc cagatgctga |
| 18301 | gggataagtt gaaagagcaa aatttccaac aaaaggtggt agatggcctg gcctctggca |
| 18361 | ttagcggggt ggtggacctg gccaaccagg cagtgcaaaa taagattaac agtaagcttg |
| 18421 | atccccgccc tcccgtagag gagcctccac cggccgtgga gacagtgtct ccagaggggc |
| 18481 | gtggcgaaaa gcgtccgcgc cccgacaggg aagaaactct ggtgacgcaa atagacgagc |
| 18541 | ctccctcgta cgaggaggca ctaaagcaag gcctgcccac cacccgtccc atcgcgccca |
| 18601 | tggctaccgg agtgctgggc cagcacacac ccgtaacgct ggacctgcct ccccccgccg |
| 18661 | acacccagca gaaacctgtg ctgccaggcc cgaccgccgt tgttgtaacc cgtcctagcc |
| 18721 | gcgcgtccct gcgccgcgcc gccagcggtc cgcgatcgtt gcggcccgta gccagtggca |
| 18781 | actggcaaag cacactgaac agcatcgtgg gtctgggggt gcaatccctg aagcgccgac |
| 18841 | gatgcttctg aatagctaac gtgtcgtatg tgtgtcatgt atgcgtccat gtcgccgcca |
| 18901 | gaggagctgc tgagccgccg cgcgcccgct ttccaagatg gctacccctt cgatgatgcc |
| 18961 | gcagtggtct tacatgcaca tctcgggcca ggacgcctcg gagtacctga gccccgggct |
| 19021 | ggtgcagttt gcccgcgcca ccgagacgta cttcagcctg aataacaagt ttagaaaccc |
| 19081 | cacggtggcg cctacgcacg acgtgaccac agaccggtcc cagcgtttga cgctgcggtt |
| 19141 | catccctgtg gaccgtgagg atactgcgta ctcgtacaag gcgcggttca ccctagctgt |
| 19201 | gggtgataac cgtgtgctgg acatggcttc cacgtacttt gacatccgcg gcgtgctgga |
| 19261 | caggggccct acttttaagc cctactctgg cactgcctac aacgccctgg ctcccaaggg |
| 19321 | tgccccaaat ccttgcgaat gggatgaagc tgctactgct cttgaaataa acctagaaga |
| 19381 | agaggacgat gacaacgaag acgaagtaga cgagcaagct gagcagcaaa aaactcacgt |
| 19441 | atttgggcag gcgccttatt ctggtataaa tattacaaag gagggtattc aaataggtgt |
| 19501 | cgaaggtcaa acacctaaat atgccgataa aacatttcaa cctgaacctc aaataggaga |
| 19561 | atctcagtgg tacgaaactg aaattaatca tgcagctggg agagtcctta aaaagactac |
| 19621 | cccaatgaaa ccatgttacg gttcatatgc aaaacccaca aatgaaaatg gagggcaagg |
| 19681 | cattcttgta aagcaacaaa atggaaagct agaaagtcaa gtggaaatgc aatttttctc |
| 19741 | aactactgag gcgaccgcag gcaatggtga taacttgact cctaaagtgg tattgtacag |
| 19801 | tgaagatgta gatatagaaa ccccagacac tcatatttct tacatgccca ctattaagga |
| 19861 | aggtaactca cgagaactaa tgggccaaca atctatgccc aacaggccta attacattgc |
| 19921 | ttttagggac aattttattg gtctaatgta ttacaacagc acgggtaata tgggtgttct |
| 19981 | ggcgggccaa gcatcgcagt tgaatgctgt tgtagatttg caagacagaa acacagagct |
| 20041 | ttcataccag cttttgcttg attccattgg tgatagaacc aggtactttt ctatgtggaa |
| 20101 | tcaggctgtt gacagctatg atccagatgt tagaattatt gaaaatcatg gaactgaaga |
| 20161 | tgaacttcca aattactgct ttccactggg aggtgtgatt aatacagaga ctcttaccaa |
| 20221 | ggtaaaacct aaaacaggtc aggaaaatgg atgggaaaaa gatgctacag aattttcaga |
| 20281 | taaaaatgaa ataagagttg gaaataattt tgccatggaa atcaatctaa atgccaacct |
| 20341 | gtggagaaat ttcctgtact ccaacatagc gctgtatttg cccgacaagc taaagtacag |
| 20401 | tccttccaac gtaaaaattt ctgataaccc aaacacctac gactacatga acaagcgagt |
| 20461 | ggtggctccc gggttagtgg actgctacat taaccttgga gcacgctggt cccttgacta |
| 20521 | tatggacaac gtcaacccat ttaaccacca ccgcaatgct ggcctgcgct accgctcaat |
| 20581 | gttgctgggc aatggtcgct atgtgccctt ccacatccag gtgcctcaga agttctttgc |
| 20641 | cattaaaaac ctccttctcc tgccgggctc atacacctac gagtggaact tcaggaagga |
| 20701 | tgttaacatg gttctgcaga gctccctagg aaatgaccta agggttgacg gagccagcat |
| 20761 | taagtttgat agcatttgcc tttacgccac cttcttcccc atggcccaca acaccgcctc |
| 20821 | cacgcttgag gccatgctta gaaacgacac caacgaccag tcctttaacg actatctctc |
| 20881 | cgccgccaac atgctctacc ctatacccgc caacgctacc aacgtgccca tatccatccc |
| 20941 | ctcccgcaac tgggcggctt tccgcggctg ggccttcacg cgccttaaga ctaaggaaac |
| 21001 | cccatcactg ggctcgggct acgaccctta ttacacctac tctggctcta taccctacct |
| 21061 | agatggaacc ttttacctca accacacctt taagaaggtg gccattacct ttgactcttc |
| 21121 | tgtcagctgg cctggcaatg accgcctgct tacccccaac gagtttgaaa ttaagcgctc |
| 21181 | agttgacggg gagggttaca acgttgccca gtgtaacatg accaaagact ggttcctggt |
| 21241 | acaaatgcta gctaactaca acattggcta ccagggcttc tatatcccag agagctacaa |
| 21301 | ggaccgcatg tactccttct ttagaaactt ccagcccatg agccgtcagg tggtggatga |
| 21361 | tactaaatac aaggactacc aacaggtggg catcctacac caacacaaca actctggatt |
| 21421 | tgttggctac cttgccccca ccatgcgcga aggacaggcc taccctgcta acttccccta |
| 21481 | tccgcttata ggcaagaccg cagttgacag cattacccag aaaaagtttc tttgcgatcg |
| 21541 | caccctttgg cgcatcccat tctccagtaa ctttatgtcc atgggcgcac tcacagacct |
| 21601 | gggccaaaac cttctctacg ccaactccgc ccacgcgcta gacatgactt ttgaggtgga |
| 21661 | tcccatggac gagcccaccc ttctttatgt tttgtttgaa gtctttgacg tggtccgtgt |
| 21721 | gcaccggccg caccgcggcg tcatcgaaac cgtgtacctg cgcacgccct tctcggccgg |
| 21781 | caacgccaca acataaagaa gcaagcaaca tcaacaacag ctgccgccat gggctccagt |
| 21841 | gagcaggaac tgaaagccat tgtcaaagat cttggttgtg ggccatattt tttgggcacc |
| 21901 | tatgacaagc gctttccagg ctttgtttct ccacacaagc tcgcctgcgc catagtcaat |
| 21961 | acggccggtc gcgagactgg gggcgtacac tggatggcct ttgcctggaa cccgcactca |
| 22021 | aaaacatgct acctctttga gccctttggc ttttctgacc agcgactcaa gcaggtttac |
| 22081 | cagtttgagt acgagtcact cctgcgccgt agcgccattg cttcttcccc cgaccgctgt |
| 22141 | ataacgctgg aaaagtccac ccaaagcgta caggggccca actcggccgc ctgtggacta |
| 22201 | ttctgctgca tgtttctcca cgcctttgcc aactggcccc aaactcccat ggatcacaac |
| 22261 | cccaccatga accttattac cggggtaccc aactccatgc tcaacagtcc ccaggtacag |
| 22321 | cccaccctgc gtcgcaacca ggaacagctc tacagcttcc tggagcgcca ctcgccctac |
| 22381 | ttccgcagcc acagtgcgca gattaggagc gccacttctt tttgtcactt gaaaaacatg |
| 22441 | taaaaataat gtactagaga cactttcaat aaaggcaaat gcttttattt gtacactctc |
| 22501 | gggtgattat ttacccccac ccttgccgtc tgcgccgttt aaaaatcaaa ggggttctgc |
| 22561 | cgcgcatcgc tatgcgccac tggcagggac acgttgcgat actggtgttt agtgctccac |
| 22621 | ttaaactcag gcacaaccat ccgcggcagc tcggtgaagt tttcactcca caggctgcgc |
| 22681 | accatcacca acgcgtttag caggtcgggc gccgatatct tgaagtcgca gttggggcct |
| 22741 | ccgccctgcg cgcgcgagtt gcgatacaca gggttgcagc actggaacac tatcagcgcc |
| 22801 | gggtggtgca cgctggccag cacgctcttg tcggagatca gatccgcgtc caggtcctcc |
| 22861 | gcgttgctca gggcgaacgg agtcaacttt ggtagctgcc ttcccaaaaa gggcgcgtgc |
| 22921 | ccaggctttg agttgcactc gcaccgtagt ggcatcaaaa ggtgaccgtg cccggtctgg |
| 22981 | gcgttaggat acagcgcctg cataaaagcc ttgatctgct taaaagccac ctgagccttt |
| 23041 | gcgccttcag agaagaacat gccgcaagac ttgccggaaa actgattggc cggacaggcc |
| 23101 | gcgtcgtgca cgcagcacct tgcgtcggtg ttggagatct gcaccacatt tcggccccac |
| 23161 | cggttcttca cgatcttggc cttgctagac tgctccttca gcgcgcgctg cccgttttcg |
| 23221 | ctcgtcacat ccatttcaat cacgtgctcc ttatttatca taatgcttcc gtgtagacac |
| 23281 | ttaagctcgc cttcgatctc agcgcagcgg tgcagccaca acgcgcagcc cgtgggctcg |
| 23341 | tgatgcttgt aggtcacctc tgcaaacgac tgcaggtacg cctgcaggaa tcgccccatc |
| 23401 | atcgtcacaa aggtcttgtt gctggtgaag gtcagctgca acccgcggtg ctcctcgttc |
| 23461 | agccaggtct tgcatacggc cgccagagct tccacttggt caggcagtag tttgaagttc |
| 23521 | gcctttagat cgttatccac gtggtacttg tccatcagcg cgcgcgcagc ctccatgccc |
| 23581 | ttctcccacg cagacacgat cggcacactc agcgggttca tcaccgtaat ttcactttcc |
| 23641 | gcttcgctgg gctcttcctc ttcctcttgc gtccgcatac cacgcgccac tgggtcgtct |
| 23701 | tcattcagcc gccgcactgt gcgcttacct cctttgccat gcttgattag caccggtggg |
| 23761 | ttgctgaaac ccaccatttg tagcgccaca tcttctcttt cttcctcgct gtccacgatt |
| 23821 | acctctggtg atggcgggcg ctcgggcttg ggagaagggc gcttcttttt cttcttgggc |
| 23881 | gcaatggcca aatccgccgc cgaggtcgat ggccgcgggc tgggtgtgcg cggcaccagc |
| 23941 | gcgtcttgtg atgagtcttc ctcgtcctcg gactcgatac gccgcctcat ccgctttttt |
| 24001 | gggggcgccc ggggaggcgg cggcgacggg gacggggacg acacgtcctc catggttggg |
| 24061 | ggacgtcgcg ccgcaccgcg tccgcgctcg ggggtggttt cgcgctgctc ctcttcccga |
| 24121 | ctggccattt ccttctccta taggcagaaa aagatcatgg agtcagtcga gaagaaggac |
| 24181 | agcctaaccg ccccctctga gttcgccacc accgcctcca ccgatgccgc caacgcgcct |
| 24241 | accaccttcc ccgtcgaggc acccccgctt gaggaggagg aagtgattat cgagcaggac |
| 24301 | ccaggttttg taagcgaaga cgacgaggac cgctcagtac caacagagga taaaaagcaa |
| 24361 | gaccaggaca acgcagaggc aaacgaggaa caagtcgggc ggggggacga aaggcatggc |
| 24421 | gactacctag atgtgggaga cgacgtgctg ttgaagcatc tgcagcgcca gtgcgccatt |
| 24481 | atctgcgacg cgttgcaaga gcgcagcgat gtgcccctcg ccatagcgga tgtcagcctt |
| 24541 | gcctacgaac gccacctatt ctcaccgcgc gtacccccca aacgccaaga aaacggcaca |
| 24601 | tgcgagccca acccgcgcct caacttctac cccgtatttg ccgtgccaga ggtgcttgcc |
| 24661 | acctatcaca tctttttcca aaactgcaag atacccctat cctgccgtgc caaccgcagc |
| 24721 | cgagcggaca agcagctggc cttgcggcag ggcgctgtca tacctgatat cgcctcgctc |
| 24781 | aacgaagtgc caaaaatctt tgagggtctt ggacgcgacg agaagcgcgc ggcaaacgct |
| 24841 | ctgcaacagg aaaacagcga aaatgaaagt cactctggag tgttggtgga actcgagggt |
| 24901 | gacaacgcgc gcctagccgt actaaaacgc agcatcgagg tcacccactt tgcctacccg |
| 24961 | gcacttaacc taccccccaa ggtcatgagc acagtcatga gtgagctgat cgtgcgccgt |
| 25021 | gcgcagcccc tggagaggga tgcaaatttg caagaacaaa cagaggaggg cctacccgca |
| 25081 | gttggcgacg agcagctagc gcgctggctt caaacgcgcg agcctgccga cttggaggag |
| 25141 | cgacgcaaac taatgatggc cgcagtgctc gttaccgtgg agcttgagtg catgcagcgg |
| 25201 | ttctttgctg acccggagat gcagcgcaag ctagaggaaa cattgcacta cacctttcga |
| 25261 | cagggctacg tacgccaggc ctgcaagatc tccaacgtgg agctctgcaa cctggtctcc |
| 25321 | taccttggaa ttttgcacga aaaccgcctt gggcaaaacg tgcttcattc cacgctcaag |
| 25381 | ggcgaggcgc gccgcgacta cgtccgcgac tgcgtttact tatttctatg ctacacctgg |
| 25441 | cagacggcca tgggcgtttg gcagcagtgc ttggaggagt gcaacctcaa ggagctgcag |
| 25501 | aaactgctaa agcaaaactt gaaggaccta tggacggcct tcaacgagcg ctccgtggcc |
| 25561 | gcgcacctgg cggacatcat tttccccgaa cgcctgctta aaaccctgca acagggtctg |
| 25621 | ccagacttca ccagtcaaag catgttgcag aactttagga actttatcct agagcgctca |
| 25681 | ggaatcttgc ccgccacctg ctgtgcactt cctagcgact ttgtgcccat taagtaccgc |
| 25741 | gaatgccctc cgccgctttg gggccactgc taccttctgc agctagccaa ctaccttgcc |
| 25801 | taccactctg acataatgga agacgtgagc ggtgacggtc tactggagtg tcactgtcgc |
| 25861 | tgcaacctat gcaccccgca ccgctccctg gtttgcaatt cgcagctgct taacgaaagt |
| 25921 | caaattatcg gtacctttga gctgcagggt ccctcgcctg acgaaaagtc cgcggctccg |
| 25981 | gggttgaaac tcactccggg gctgtggacg tcggcttacc ttcgcaaatt tgtacctgag |
| 26041 | gactaccacg cccacgagat taggttctac gaagaccaat cccgcccgcc aaatgcggag |
| 26101 | cttaccgcct gcgtcattac ccagggccac attcttggcc aattgcaagc catcaacaaa |
| 26161 | gcccgccaag agtttctgct acgaaaggga cggggggttt acttggaccc ccagtccggc |
| 26221 | gaggagctca acccaatccc cccgccgccg cagccctatc agcagcagcc gcgggccctt |
| 26281 | gcttcccagg atggcaccca aaaagaagct gcagctgccg ccgccaccca cggacgagga |
| 26341 | ggaatactgg gacagtcagg cagaggaggt tttggacgag gaggaggagg acatgatgga |
| 26401 | agactgggag agcctagacg aggaagcttc cgaggtcgaa gaggtgtcag acgaaacacc |
| 26461 | gtcaccctcg gtcgcattcc cctcgccggc gccccagaaa tcggcaaccg gttccagcat |
| 26521 | ggctacaacc tccgctcctc aggcgccgcc ggcactgccc gttcgccgac ccaaccgtag |
| 26581 | atgggacacc actggaacca gggccggtaa gtccaagcag ccgccgccgt tagcccaaga |
| 26641 | gcaacaacag cgccaaggct accgctcatg gcgcgggcac aagaacgcca tagttgcttg |
| 26701 | cttgcaagac tgtgggggca acatctcctt cgcccgccgc tttcttctct accatcacgg |
| 26761 | cgtggccttc ccccgtaaca tcctgcatta ctaccgtcat ctctacagcc catactgcac |
| 26821 | cggcggcagc ggcagcggca gcaacagcag cggccacaca gaagcaaagg cgaccggata |
| 26881 | gcaagactct gacaaagccc aagaaatcca cagcggcggc agcagcagga ggaggagcgc |
| 26941 | tgcgtctggc gcccaacgaa cccgtatcga cccgcgagct tagaaacagg atttttccca |
| 27001 | ctctgtatgc tatatttcaa cagagcaggg gccaagaaca agagctgaaa ataaaaaaca |
| 27061 | ggtctctgcg atccctcacc cgcagctgcc tgtatcacaa aagcgaagat cagcttcggc |
| 27121 | gcacgctgga agacgcggag gctctcttca gtaaatactg cgcgctgact cttaaggact |
| 27181 | agtttcgcgc cctttctcaa atttaagcgc gaaaactacg tcatctccag cggccacacc |
| 27241 | cggcgccagc acctgtcgtc agcgccatta tgagcaagga aattcccacg ccctacatgt |
| 27301 | ggagttacca gccacaaatg ggacttgcgg ctggagctgc ccaagactac tcaacccgaa |
| 27361 | taaactacat gagcgcggga ccccacatga tatcccgggt caacggaatc cgcgcccacc |
| 27421 | gaaaccgaat tctcttggaa caggcggcta ttaccaccac acctcgtaat aaccttaatc |
| 27481 | cccgtagttg gcccgctgcc ctggtgtacc aggaaagtcc cgctcccacc actgtggtac |
| 27541 | ttcccagaga cgcccaggcc gaagttcaga tgactaactc aggggcgcag cttgcgggcg |
| 27601 | gctttcgtca cagggtgcgg tcgcccgggc agggtataac tcacctgaca atcagagggc |
| 27661 | gaggtattca gctcaacgac gagtcggtga gctcctcgct tggtctccgt ccggacggga |
| 27721 | catttcagat cggcggcgcc ggccgtcctt cattcacgcc tcgtcaggca atcctaactc |
| 27781 | tgcagacctc gtcctctgag ccgcgctctg gaggcattgg aactctgcaa tttattgagg |
| 27841 | agtttgtgcc atcggtctac tttaacccct tctcgggacc tcccggccac tatccggatc |
| 27901 | aatttattcc taactttgac gcggtaaagg actcggcgga cggctacgac tgaatgttaa |
| 27961 | gtggagaggc agagcaactg cgcctgaaac acctggtcca ctgtcgccgc cacaagtgct |
| 28021 | ttgcccgcga ctccggtgag ttttgctact ttgaattgcc cgaggatcat atcgagggcc |
| 28081 | cggcgcacgg cgtccggctt accgcccagg gagagcttgc ccgtagcctg attcgggagt |
| 28141 | ttacccagcg ccccctgcta gttgagcggg acaggggacc ctgtgttctc actgtgattt |
| 28201 | gcaactgtcc taaccttgga ttacatcaag atctttgttg ccatctctgt gctgagtata |
| 28261 | ataaatacag aaattaaaat atactggggc tcctatcgcc atcctgtaaa cgccaccgtc |
| 28321 | ttcacccgcc caagcaaacc aaggcgaacc ttacctggta cttttaacat ctctccctct |
| 28381 | gtgatttaca acagtttcaa cccagacgga gtgagtctac gagagaacct ctccgagctc |
| 28441 | agctactcca tcagaaaaaa caccaccctc cttacctgcc gggaacgtac gagtgcgtca |
| 28501 | ccggccgctg caccacacct accgcctgac cgtaaaccag actttttccg gacagacctc |
| 28561 | aataactctg tttaccagaa caggaggtga gcttagaaaa cccttagggt attaggccaa |
| 28621 | aggcgcagct actgtggggt ttatgaacaa ttcaagcaac tctacgggct attctaattc |
| 28681 | aggtttctct agaatcgggg ttggggttat tctctgtctt gtgattctct ttattcttat |
| 28741 | actaacgctt ctctgcctaa ggctcgccgc ctgctgtgtg cacatttgca tttattgtca |
| 28801 | gctttttaaa cgctggggtc gccacccaag atgattaggt acataatcct aggtttactc |
| 28861 | acccttgcgt cagcccacgg taccacccaa aaggtggatt ttaaggagcc agcctgtaat |
| 28921 | gttacattcg cagctgaagc taatgagtgc accactctta taaaatgcac cacagaacat |
| 28981 | gaaaagctgc ttattcgcca caaaaacaaa attggcaagt atgctgttta tgctatttgg |
| 29041 | cagccaggtg acactacaga gtataatgtt acagttttcc agggtaaaag tcataaaact |
| 29101 | tttatgtata cttttccatt ttatgaaatg tgcgacatta ccatgtacat gagcaaacag |
| 29161 | tataagttgt ggcccccaca aaattgtgtg gaaaacactg gcactttctg ctgcactgct |
| 29221 | atgctaatta cagtgctcgc tttggtctgt accctactct atattaaata caaaagcaga |
| 29281 | cgcagcttta ttgaggaaaa gaaaatgcct taatttacta agttacaaag ctaatgtcac |
| 29341 | cactaactgc tttactcgct gcttgcaaaa caaattcaaa aagttagcat tataattaga |
| 29401 | ataggattta aaccccccgg tcatttcctg ctcaatacca ttcccctgaa caattgactc |
| 29461 | tatgtgggat atgctccagc gctacaacct tgaagtcagg cttcctggat gtcagcatct |
| 29521 | gactttggcc agcacctgtc ccgcggattt gttccagtcc aactacagcg acccacccta |
| 29581 | acagagatga ccaacacaac caacgcggcc gccgctaccg gacttacatc taccacaaat |
| 29641 | acaccccaag tttctgcctt tgtcaataac tgggataact tgggcatgtg gtggttctcc |
| 29701 | atagcgctta tgtttgtatg ccttattatt atgtggctca tctgctgcct aaagcgcaaa |
| 29761 | cgcgcccgac cacccatcta tagtcccatc attgtgctac acccaaacaa tgatggaatc |
| 29821 | catagattgg acggactgaa acacatgttc ttttctctta cagtatgatt aaatgagaca |
| 29881 | tgattcctcg agtttttata ttactgaccc ttgttgcgct tttttgtgcg tgctccacat |
| 29941 | tggctgcggt ttctcacatc gaagtagact gcattccagc cttcacagtc tatttgcttt |
| 30001 | acggatttgt caccctcacg ctcatctgca gcctcatcac tgtggtcatc gcctttatcc |
| 30061 | agtgcattga ctgggtctgt gtgcgctttg catatctcag acaccatccc cagtacaggg |
| 30121 | acaggactat agctgagctt cttagaattc tttaattatg aaatttactg tgacttttct |
| 30181 | gctgattatt tgcaccctat ctgcgttttg ttccccgacc tccaagcctc aaagacatat |
| 30241 | atcatgcaga ttcactcgta tatggaatat tccaagttgc tacaatgaaa aaagcgatct |
| 30301 | ttccgaagcc tggttatatg caatcatctc tgttatggtg ttctgcagta ccatcttagc |
| 30361 | cctagctata tatccctacc ttgacattgg ctggaaacga atagatgcca tgaaccaccc |
| 30421 | aactttcccc gcgcccgcta tgcttccact gcaacaagtt gttgccggcg gctttgtccc |
| 30481 | agccaatcag cctcgcccca cttctcccac ccccactgaa atcagctact ttaatctaac |
| 30541 | aggaggagat gactgacacc ctagatctag aaatggacgg aattattaca gagcagcgcc |
| 30601 | tgctagaaag acgcagggca gcggccgagc aacagcgcat gaatcaagag ctccaagaca |
| 30661 | tggttaactt gcaccagtgc aaaaggggta tcttttgtct ggtaaagcag gccaaagtca |
| 30721 | cctacgacag taataccacc ggacaccgcc ttagctacaa gttgccaacc aagcgtcaga |
| 30781 | aattggtggt catggtggga gaaaagccca ttaccataac tcagcactcg gtagaaaccg |
| 30841 | aaggctgcat tcactcacct tgtcaaggac ctgaggatct ctgcaccctt attaagaccc |
| 30901 | tgtgcggtct caaagatctt attcccttta actaataaaa aaaaataata aagcatcact |
| 30961 | tacttaaaat cagttagcaa atttctgtcc agtttattca gcagcacctc cttgccctcc |
| 31021 | tcccagctct ggtattgcag cttcctcctg gctgcaaact ttctccacaa tctaaatgga |
| 31081 | atgtcagttt cctcctgttc ctgtccatcc gcacccacta tcttcatgtt gttgcagatg |
| 31141 | aagcgcgcaa gaccgtctga agataccttc aaccccgtgt atccatatga cacggaaacc |
| 31201 | ggtcctccaa ctgtgccttt tcttactcct ccctttgtat cccccaatgg gtttcaagag |
| 31261 | agtccccctg gggtactctc tttgcgccta tccgaacctc tagttacctc caatggcatg |
| 31321 | cttgcgctca aaatgggcaa cggcctctct ctggacgagg ccgggaactt gacctcacag |
| 31381 | aacgtttata gtcgtttaaa tgaaattgac actaaacaga caacagttga gtctgacatt |
| 31441 | agtgctatta agacctcaat aggatatcca ggaaataatt cgattatcac gagtgttaat |
| 31501 | acaaacactg ataatattgc atctattaat ttagagctaa atcaaagtgg aggtattaaa |
| 31561 | cagcgtttaa ccgttattga aacttccatt ggttcagatg atattccttc gagtattaaa |
| 31621 | ggtcaaatca aagataatac aacttcaatc gaatctctaa atggaatcgt cggtgaaaac |
| 31681 | acttcatctg gcttaagagc gaatgtttca tggttaaacc aaattgttgg aactgattct |
| 31741 | agcggtggac aaccttctcc tcctgggtct cttttaaacc gagtttctac aattgaaact |
| 31801 | tctgtttcag gcttgaataa cgatgttcaa aacctacaag tagagattgg taataacagc |
| 31861 | acaggaatta aagggcaagt tgtagcgtta aatactttag taaatggaac taatccaaac |
| 31921 | ggttcaactg ttgaagagcg cggattaacc aattcaataa aagctaacga aactaacatt |
| 31981 | gcatcagtta cacaagaagt gaatacagct aaaggcaata tatcttcttt acaaggtgat |
| 32041 | gttcaagctc tccaagaagc cggttatatt cctgaagctc caagagatgg gcaagcttac |
| 32101 | gttcgtaaag atggcgaatg ggtattgctt tctacctttt tatcgccggc aggtggaggc |
| 32161 | ggttcaggcg gaggtggctc tggcggtggc ggatccgcta tcgttatgac ccagccgagt |
| 32221 | tctctgtctg tatcactggg tgaacgtgtt accatcagct gccgcgcgtc ccagagcctg |
| 32281 | gaaaacagca atggtaacac cttcctgaat tggtatcagc aaaagcctgg ccaaccgccg |
| 32341 | aaactgctga tctaccgtgt ttctaaccgt ttctctggtg ttccggaccg tttctcgggt |
| 32401 | agcggcagcg gtaccgactt cactctgacc atctcttctg tacaggctga agatctggct |
| 32461 | gtttactact gtctgcaagt tacccacgta ccgtacacct ttggcgctgg caccaaactg |
| 32521 | gagctcaagg gtggcggtgg ctcgggcggt ggtgggtcgg gtggcggcgg atctgaagtt |
| 32581 | aaactgcagc agagcggggc tgagcttgtg aagcccgggg gttcagtgaa gctgtcctgc |
| 32641 | aaggcttctg gctacacctt caccaccaac tacaattgga actgggtgaa gcagaggcct |
| 32701 | ggacaaggtc tcgagtggat tggatacatt cgttatgacg gtacctctga atacaccccg |
| 32761 | tccctgaaga acaaggccac actgactgta gacaaatcgt ccagcacagc ctacatgcag |
| 32821 | ctcagcagcc tgacatctga ggactctgcg gtctattatt gtgctcgtct ggactactgg |
| 32881 | ggccaaggga ccacgctcac cgtctcctca catcatcacc atcaccacta ataaatgaat |
| 32941 | cgtttgtgtt atgtttcaac gtgtttattt ttcaattgca gaaaatttca agtcattttt |
| 33001 | cattcagtag tatagcccca ccaccacata gcttatacag atcaccgtac cttaatcaaa |
| 33061 | ctcacagaac cctagtattc aacctgccac ctccctccca acacacagag tacacagtcc |
| 33121 | tttctccccg gctggcctta aaaagcatca tatcatgggt aacagacata ttcttaggtg |
| 33181 | ttatattcca cacggtttcc tgtcgagcca aacgctcatc agtgatatta ataaactccc |
| 33241 | cgggcagctc acttaagctc atgtcgctgt ccagctgctg agccacaggc tgctgtccaa |
| 33301 | cttgcggttg cttaacgggc ggcgaaggag aagtccacgc ctacatgggg gtagagtcat |
| 33361 | aatcgtgcat caggataggg cggtggtgct gcagcagcgc gcgaataaac tgctgccgcc |
| 33421 | gccgctccgt cctgcaggaa tacaacatgg cagtggtctc ctcagcgatg attcgcaccg |
| 33481 | cccgcagcat aaggcgcctt gtcctccggg cacagcagcg caccctgatc tcacttaaat |
| 33541 | cagcacagta actgcagcac agcaccacaa tattgttcaa aatcccacag tgcaaggcgc |
| 33601 | tgtatccaaa gctcatggcg gggaccacag aacccacgtg gccatcatac cacaagcgca |
| 33661 | ggtagattaa gtggcgaccc ctcataaaca cgctggacat aaacattacc tcttttggca |
| 33721 | tgttgtaatt caccacctcc cggtaccata taaacctctg attaaacatg gcgccatcca |
| 33781 | ccaccatcct aaaccagctg gccaaaacct gcccgccggc tatacactgc agggaaccgg |
| 33841 | gactggaaca atgacagtgg agagcccagg actcgtaacc atggatcatc atgctcgtca |
| 33901 | tgatatcaat gttggcacaa cacaggcaca cgtgcataca cttcctcagg attacaagct |
| 33961 | cctcccgcgt tagaaccata tcccagggaa caacccattc ctgaatcagc gtaaatccca |
| 34021 | cactgcaggg aagacctcgc acgtaactca cgttgtgcat tgtcaaagtg ttacattcgg |
| 34081 | gcagcagcgg atgatcctcc agtatggtag cgcgggtttc tgtctcaaaa ggaggtagac |
| 34141 | gatccctact gtacggagtg cgccgagaca accgagatcg tgttggtcgt agtgtcatgc |
| 34201 | caaatggaac gccggacgta gtcatatttc ctgaagcaaa accaggtgcg ggcgtgacaa |
| 34261 | acagatctgc gtctccggtc tcgccgctta gatcgctctg tgtagtagtt gtagtatatc |
| 34321 | cactctctca aagcatccag gcgccccctg gcttcgggtt ctatgtaaac tccttcatgc |
| 34381 | gccgctgccc tgataacatc caccaccgca gaataagcca cacccagcca acctacacat |
| 34441 | tcgttctgcg agtcacacac gggaggagcg ggaagagctg gaagaaccat gttttttttt |
| 34501 | ttattccaaa agattatcca aaacctcaaa atgaagatct attaagtgaa cgcgctcccc |
| 34561 | tccggtggcg tggtcaaact ctacagccaa agaacagata atggcatttg taagatgttg |
| 34621 | cacaatggct tccaaaaggc aaacggccct cacgtccaag tggacgtaaa ggctaaaccc |
| 34681 | ttcagggtga atctcctcta taaacattcc agcaccttca accatgccca aataattctc |
| 34741 | atctcgccac cttctcaata tatctctaag caaatcccga atattaagtc cggccattgt |
| 34801 | aaaaatctgc tccagagcgc cctccacctt cagcctcaag cagcgaatca tgattgcaaa |
| 34861 | aattcaggtt cctcacagac ctgtataaga ttcaaaagcg gaacattaac aaaaataccg |
| 34921 | cgatcccgta ggtcccttcg cagggccagc tgaacataat cgtgcaggtc tgcacggacc |
| 34981 | agcgcggcca cttccccgcc aggaaccttg acaaaagaac ccacactgat tatgacacgc |
| 35041 | atactcggag ctatgctaac cagcgtagcc ccgatgtaag ctttgttgca tgggcggcga |
| 35101 | tataaaatgc aaggtgctgc tcaaaaaatc aggcaaagcc tcgcgcaaaa aagaaagcac |
| 35161 | atcgtagtca tgctcatgca gataaaggca ggtaagctcc ggaaccacca cagaaaaaga |
| 35221 | caccattttt ctctcaaaca tgtctgcggg tttctgcata aacacaaaat aaaataacaa |
| 35281 | aaaaacattt aaacattaga agcctgtctt acaacaggaa aaacaaccct tataagcata |
| 35341 | agacggacta cggccatgcc ggcgtgaccg taaaaaaact ggtcaccgtg attaaaaagc |
| 35401 | accaccgaca gctcctcggt catgtccgga gtcataatgt aagactcggt aaacacatca |
| 35461 | ggttgattca tcggtcagtg ctaaaaagcg accgaaatag cccgggggaa tacatacccg |
| 35521 | caggcgtaga gacaacatta cagcccccat aggaggtata acaaaattaa taggagagaa |
| 35581 | aaacacataa acacctgaaa aaccctcctg cctaggcaaa atagcaccct cccgctccag |
| 35641 | aacaacatac agcgcttcac agcggcagcc taacagtcag ccttaccagt aaaaaagaaa |
| 35701 | acctattaaa aaaacaccac tcgacacggc accagctcaa tcagtcacag tgtaaaaaag |
| 35761 | ggccaagtgc agagcgagta tatataggac taaaaaatga cgtaacggtt aaagtccaca |
| 35821 | aaaaacaccc agaaaaccgc acgcgaacct acgcccagaa acgaaagcca aaaaacccac |
| 35881 | aacttcctca aatcgtcact tccgttttcc cacgttacgt aacttcccat tttaagaaaa |
| 35941 | ctacaattcc caacacatac aagttactcc gccctaaaac ctacgtcacc cgccccgttc |
| 36001 | ccacgccccg cgccacgtca caaactccac cccctcatta tcatattggc ttcaatccaa |
| 36061 | aataaggtat attattgatg atg |
| | * * * |

Having thus described in detail advantageous embodiments of the present invention, it is to be understood that the invention defined by the above paragraphs is not to be limited to particular details set forth in the above description as many apparent variations thereof are possible without departing from the spirit or scope of the present invention.

1. An adenovirus (Ad) modified by replacing a native capsid protein fiber with a fiber replacement protein, wherein the fiber replacement protein comprises:
(a) an amino-terminal portion comprising the native capsid protein fiber amino terminus;
(b) a trimeric substitute for a fiber shaft knob of the native capsid protein fiber; and
(c) a carboxy-terminal portion comprising a stabilized single chain antibody (scFv) ligand.

2. The adenovirus of claim 1, wherein the trimeric substitute retains trimerism when a sequence encoding the stabilized scFv ligand is incorporated into the carboxy-terminus.

3. The adenovirus of claim 1, wherein the fiber replacement protein is soluble.

4. The adenovirus of claim 1, wherein the trimeric substitute is a T4 bacteriophage fibritin protein.

5. The adenovirus of claim 1, wherein the trimeric substitute comprises an isoleucine trimerization motif.

6. The adenovirus of claim 1, wherein the trimeric substitute comprises a neck region peptide from human lung surfactant D.

7. The adenovirus of claim 1, wherein the adenovirus comprises a transgene.

8. The adenovirus of claim 7, wherein the transgene is a herpes simplex virus thymidine kinase gene.

9. The adenovirus of claim 1, wherein the stabilized scFv ligand comprises mutations in the scFv CDR regions.

10. The adenovirus of claim 1, wherein the stabilized scFv ligand is an anti-CD40 scFv.

11. An adenoviral vector comprising the adenovirus of claim 1.

12. The vector of claim 11 wherein the adenovirus is operatively linked to a non-viral promoter.

13. A transformed host cell comprising the vector of claim 11.

14. The transformed host cell of claim 13, wherein the vector is introduced into the cell by transfection, electroporation or transformation.

15. A method for preparing a transformed cell expressing the adenovirus of claim 1 comprising:
(a) transfecting, electroporating or transforming a cell with the adenovirus of claim 1 to produce a transformed host cell and
(b) maintaining the transformed host cell under biological conditions sufficient for expression of the adenovirus in the host cell.

16. A method for inhibiting tumor cell growth in a subject in need thereof comprising administering to the subject in need thereof a therapeutically effective amount of the adenovirus of claim 1 wherein the scFv ligand targets the tumor cell such that the adenovirus infects the tumor cells and thereby inhibits tumor cell growth in the subject.

17. The method of claim 16 wherein the adenovirus further comprises a transgene.

18. The method of claim 17 wherein the transgene is a herpes simplex virus thymidine kinase gene.

19. The method of claim 17 further comprising administrating ganciclovir.

20. An adenovirus comprising the nucleotide base sequence of SEQ ID NO. 21.
